(19) United States
(12) Patent Application Publication
(10) Pub. No.: US 2013/0108618 A1
(43) Pub. Date: May 2, 2013

(54) METHODS AND COMPOSITIONS RELATED TO INTRACELLULAR NEUTRALIZATION BY IgG

(71) Applicant: University of Maryland, College Park, MD (US)

(72) Inventor: Xiaoping Zhu, Clarksville, MD (US)

(73) Assignee: University of Maryland, College Park, MD (US)

(21) Appl. No.: 13/663,468

(22) Filed: Oct. 29, 2012

Related U.S. Application Data
(60) Provisional application No. 61/553,024, filed on Oct. 28, 2011.

(51) Int. Cl. A61K 39/395; G01N 33/53; C07K 16/12; C07K 16/30; C07K 16/18; C07K 16/08

(52) U.S. Cl. CPC A61K 39/395; C07K 16/18; C07K 16/08; C07K 16/12; C07K 16/30; G01N 33/53
USPC 424/130.1; 530/387.1; 530/389.1; 530/389.4; 530/389.5; 530/389.7; 435/5

(57) ABSTRACT

Disclosed are compositions, antibodies, and methods for binding intracellular antigens.

This application claims benefit of U.S. Provisional Application No. 61/553,024 filed on Oct. 28, 2011, which is incorporated herein in its entirety. This invention was made with government support under R01AI065892, R21AI067965, R21AI073139, DK56597, and R37AI041239-06A1, awarded by the National Institutes of Health. The government has certain rights in the invention.

### BACKGROUND

1. Antibodies, including mucosal antibodies, provide a primary line of defense against pathogen invasion. Most pathogens (>90%) initiate their infections at the apical domain, although the basolateral domain is also targeted in some cases. Receptor-mediated endocytosis of viruses and post-endocytic membrane fusion has long been accepted as a cell entry mechanism for many viruses. For example, influenza virus replication begins with hemagglutinin (HA) binding to the cellular receptor in apical surface of airway epithelial cells, after which the viruses are internalized inro endosomes. Traditionally, IgG is thought to function extracellular by preventing virion attachment of or penetration into polarized epithelium. Due to the traditional notion vaccine, as well as therapeutic and neutralizing antibody design strategy has only focused on antigens thought to be targeted by naturally occurring antibodies. That is antigens on extracellular pathogens. However, such design strategy in effect means that only the small number of antigens that are expressed on the surface of a pathogen in an extracellular environment are targeted. Moreover, the vast majority of antigens, primarily available in the intracellular environment, are neglected. What are needed are antibodies and vaccines that can target antigens that are available to the intracellular environment.

### SUMMARY

2. Disclosed are methods and compositions related to antibodies specific for a non-surface expressed antigen or an antigenic determinant that is only accessible to an antibody through a conformational change of the antigen. In one aspect, the disclosed compositions and antibodies can be used as part of a vaccine or passive immunotherapy.

3. Also disclosed herein are method of treating or inhibiting a disease or condition comprising administering to a subject one or more of the antibodies disclosed herein.

4. In another aspect, disclosed herein are methods of diagnosing a disease or condition or detecting exposure to an antigen in a subject comprising obtaining a tissue sample from the subject and contacting the tissue with one or more antibodies of claim 1, wherein the one or more antibodies comprise a detectable label, wherein detection of the one or more antibodies indicates the subject has the disease or condition or has been exposed to the pathogen.

### BRIEF DESCRIPTION OF THE DRAWINGS

5. The accompanying drawings, which are incorporated in and constitute a part of this specification, illustrate several embodiments and together with the description illustrate the disclosed compositions and methods.

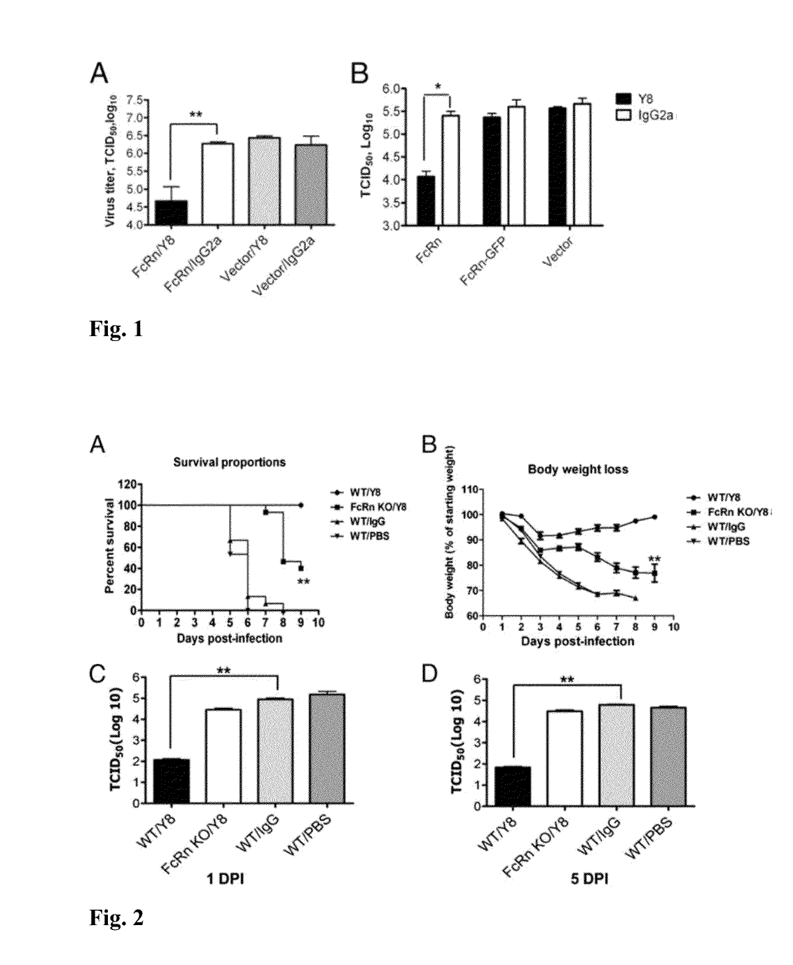

6. FIG. 1 shows the neutralization of influenza PR8 virus in MDCK-FcRn cells by Y8 mAb. Cells (1×10⁵/well) were grown in a 0.4-mm transwell insert and allowed to polarize. FIG. 1A shows the neutralization of PR8 virus by Y8 transcytosis. Y8 mAb or IgG2a isotype (400 mg/mL) was added to the basolateral chamber for 2 h at 37° C.; subsequently, PR8 virus (100 pfu/cell) was added to the apical chamber for 1.5 h at 4° C., then switched to 37° C. for 45 min. Cells in both chambers were completely washed of residual IgG to remove adherent virus particles. Monolayers were then incubated for an additional 24 h at 37° C. The amount of PR8 virus in the apical medium was analyzed by TCID₅₀ assay. FIG. 1B shows that the neutralization of PR8 virus by Y8 mAb is dependent on IgG transcytosis. Y8 mAb (400 mg/mL) was added to the basolateral chamber of MDCK-FcRn, MDCK-FcRn-GFP, or control cells for 2 h at 37° C. PR8 virus was subsequently added to the apical side for 1.5 h at 4° C., and then cells were switched to 37° C. for another 45 min to allow for infection. The remaining procedures were performed as in 1B.

7. FIG. 2 shows that PR8 HA-specific Y8 mAb protected mice from virus infection. (A and B) Severity of infection in mice challenged with PR8 virus. Groups of five WT and FcRn-KO mice were intraperitoneally injected with 100 mg Y8 mAb or control IgG. One group of five mice was mock-injected with PBS solution. Four hours later, mice were intranasally challenged with 500 pfu of PR8 virus. The mice were monitored for 10 d. FcRn-KO mice were injected daily with 25-57.5 mg Y8 or control IgG to compensate for IgG catabolism. FIG. 2A shows the survival rate was assessed by recording whether the mice died from the infection. Percentage of mice protected on the indicated days was calculated as the number of mice surviving divided by the number of mice in each group and averaged over three similar experiments (n=15). The mice were also weighed daily to monitor illness, as defined by percent weight loss (32B). For virus titration, lungs were harvested at day 1 (2C) or day 5 (2D) after infection and homogenized. The amount of PR8 virus in the supernatant was analyzed by TCID₅₀. Data shown are the means of three independent experiments, with five mice per group (**P<0.01).

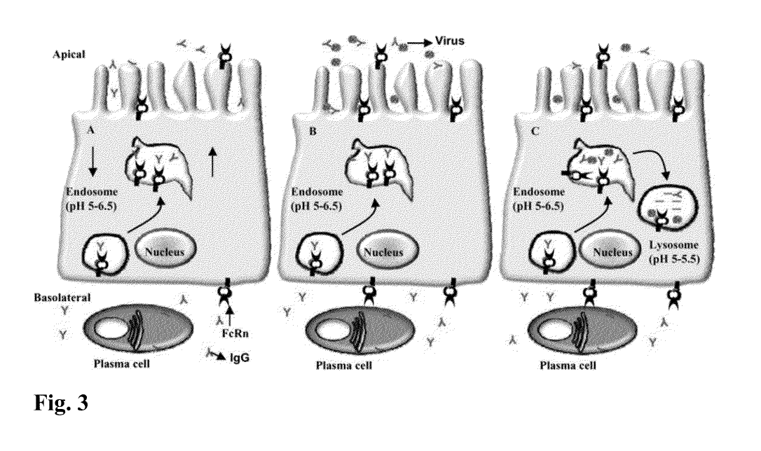

8. FIG. 3 shows a model for IgG-mediated intracellular neutralization by FcRn in polarized epithelial cells. FIG. 4A shows that FcRn transports IgG bidirectionally. FIG. 4B shows that IgG is transcytosed and secreted into the lumen, where it can combine antigens to form immune complexes. FIG. 4C shows that in a cell that has been infected by a virus, a transcytotic vesicle containing antiviral IgG has the opportunity to meet virus. IgG neutralizes the virus inside vesicles and therefore aborts viral replication by delivery of these particles to lysosomes for degradation.

### DETAILED DESCRIPTION

9. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

10. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

11. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

12. In this specification and in the claims which follow, reference will be made to a number of terms which shall be defined to have the following meanings:

13. “Optional” or “optionally” means that the subsequently described event or circumstance may or may not occur, and that the description includes instances where said event or circumstance occurs and instances where it does not.

14. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

15. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

16. Disclosed herein it is shown that the neonatal Fc receptor (FcRn) shuttles the IgG antibody across mucosal surfaces. The FcRn was initially thought to transport maternal IgG to human fetuses through the placenta or to newborns via the intestine in neonatal life. It is shown herein that FcRn can function beyond neonatal life because of the functional expression of FcRn in adult tissues. By transcytosing IgG across the vascular endothelium at all stages of life, FcRn ensures the extravascular bioavailability of IgG. Finally, by transcytosing IgG across the mucosal epithelium, FcRn provides a line of humoral defense at the mucosal surfaces. The functional discovery of FcRn explains why IgG, but not IgA, is a major Ig in the lung and genital tract.

17. In addition to its transcytotic function, FcRn plays a role in serum IgG homeostasis by recycling IgG away from a catabolic pathway in vascular endothelium, thus extending its lifespan in circulation and ensuring long-lasting protective immunity after infection. A hallmark of FcRn is that it binds IgG at acidic pH (<6.5) and releases IgG at neutral or higher pH. In the majority of cell types, FcRn resides primarily in early acidic endosomal vesicles; FcRn binds to IgG that enters the cell by pinocytosis or endocytosis. Subsequently, FcRn efficiently recycles IgG back to the plasma membrane or transcytoses it to the opposite plasma membrane, where the near-neutral pH of the extracellular environment causes IgG release from FcRn. Any pinocytosed or endocytosed proteins, including IgG, that are not rescued in this manner are efficiently trafficked to the lysosomes for degradation.

18. Epithelial monolayers lining the mucosal surfaces polarize into two separate plasma membrane domains, the apical and basolateral, which are separated by intercellular tight junctions at the apical poles. The vast mucosal surfaces represent major sites of potential attack by invading pathogens. Most pathogens (>90%) initiate their infections at the apical domain, although the basolateral domain is also targeted in some cases. Receptor-mediated endocytosis of viruses and postendocytic membrane fusion has long been accepted as a cell entry mechanism for many viruses. For enveloped viruses, fusion of the viral lipid bilayer with the membrane of an acidic endosome is generally catalyzed by a “fusion protein” on the viral surface. Influenza A virus infection begins with the interaction of virions with cell surface sialic acid residues primarily mediated by hemagglutinin (HA). After binding virions are internalized through endocytic pathways the acidic pH within the endosomes induces a conformational change in the viral proteins such as, HA, which in turn triggers fusion between the viral envelope and the endosomal membranes. Subsequently, the low pH induces further conformational changes in the viral matrix and viral ribonucleoprotein (vRNP) which are ejected into the cytoplasm and the vRNP is actively imported into the nucleus. Viral proteins produced in the cytoplasm assemble with replicated viral RNA and bud from the cell membrane.

19. The internalization of the pathogen and subsequent conformational changes makes available antigens that are not present in the extracellular milieu. Accordingly, disclosed herein are antibodies specific for a non-surface expressed antigen or an antigenic determinant that is only accessible to an antibody through a conformational change of the antigen. The antibodies can be isolated or part of a composition such as a vaccine, passive immunization, or passive immunotherapy. It is understood and herein contemplated that the antibodies disclosed herein either isolated or as part of a vaccine or larger composition are of the IgG isotype to facilitate internalization by FcRn. It is further understood that the disclose antibodies can be neutralizing antibodies.

20. “Antigen” means any native or foreign substance that is capable of eliciting an immune response. Preferably, the antigen will elicit an antibody, plasma cell, plasmablast, or B-cell response. Such antigens can include but are not limited to peptides and/or proteins from a subject, virus, bacteria, yeast, or parasite, including but not limited to toxins. Antigens can also include vaccines (e.g., peptides, proteins, killed pathogens, or attenuated pathogens administered in a pharmaceutically acceptable carrier either prophylactically or therapeutically), bio-warfare agents, and native peptides, polypeptides, and proteins.

21. Since viral, bacterial, fungal, and parasitic antigens, including viral antigens such as, HA, vary among strains and are continuously changing, a vaccine produced against one strain will be less effective or ineffective against other strains. This is highly challenging, because multiple strains circulate in the population each flu season, and new strains are continually emerging. Indeed, availability of strain-matched vaccines usually lags behind these antigenic changes. For example, the ultimate goal of developing a “universal” flu vaccine that protects against almost all strains of flu is highly desirable and needed. In one aspect, disclosed herein are antibodies, vaccines, and compositions that target conserved parts of viral, bacterial, fungal, parasitic, and cancer antigens. Consequently, these antigens can be recognized by the immune system from strain to strain.

22. It is understood and herein contemplated that the antibodies disclosed herein can bind to antigens that are internal or otherwise unavailable when a virus, bacteria, fungi, or parasite is in the extracellular environment. Thus, in one aspect, disclosed herein are antibodies specific for an antigen that is present in or on the surface of a pathogen or encoded by a pathogen.

### Anti-viral Antibodies

23. In one aspect, the pathogen can be a virus and the antigen a viral antigen. Disclosed herein are antibodies and compositions comprising said antibodies, such as, for example, vaccines, passive immunotherapy, and passive immunizations wherein the antibody is specific for a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A (including H1N1 or other Swine H1), Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In a further aspect, the viral antigen can be a viral nonstructural protein, strucutural protein, regulatory protein or accessory protein. Thus, the viral antigen can be a viral glycoprotein (GP), portal protein, tegument protein, capsid protein, DNA polymerase, RNA polymerase, reverse transcriptase, protease, integrase, DNA-binding protein, nucleoprotein (NP), nuclear matric protein, envelope protein (ENV), nuclear antigen, membrane protein, proteins encoded by viral early genes, group specific antigen (gag) protein, hemagglutinin (HA), neuraminidase (NA), or matrix protein. Specific examples of viral antigens include but are not limited to ENV, GP160 (HIV) GP120 (HIV), GP41 (HIV), EBNA-1, EBNA-2, EBNA-3, LMP-1, LMP-2, E1, E2, E3, E4, E5, E6, E7, NSP1, NSP2, NSP3, NSP4, NSP5, NSP10, NSP14, NSP15, NSP16, NSP29, G35P, G38P, G39P, zygocin protein, VP5 protein, 3AB protein, L4-22K protein, L4-100K protein, ORF 17 protein, S7 protein, S9 protein, S10 protein, HBXIP protein, UL3.5 protein, virus-infected-associated antigen protein, 3ABC protein, Cng protein, 2 BC protein, p58 protein, A40R protein, vpu protein, VPX protein, BPLF1 protein, NEF protein, SGTA protein, UL102 protein, p121 protein, VP35 protein, SPP1 Pac region protein, pX protein, N protein, agnoprotein, sigma NS protein, phage repressor proteins, U(S)₃ protein kinase, ToxR protein, LexA protein, lambda CI repressor protein, Mu Ner protein, and Tat proteins.

### Anti-bacterial Antibodies

24. Similarly, the pathogen can be a bacteria and the antigen a bacterial antigen. Disclosed herein are antibodies and compositions comprising said antibodies, such as, for example, vaccines, passive immunotherapy, and passive immunizations wherein the antibody is specific for a bacterial antigen from a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In another apsect, the antigen comprises a bacterial surface protein including but not limited to bacterial oligosaccharide, polysaccharide, or lipopolysaccharide; a protein associated with fimbrial structure and biogenesis, antimicrobial resistance, heavy metal transport, bacterial adhesion, extracytoplasmic substrate trafficking, or secreted hydrolases; exopolysaccharide; humic acid; N-acetylmuramic acid (NAM); N-acetylglucosamine (NAG); teichoic acids including ribitol teichoic acid and glycerol teichoic acid; O-antigen; Lipid A; pilin proteins; Porin; MA0829; or SbsB. In yet another aspect, the antigen can be a a component of a microbial biofilm, examples of which include but are not limited to exopolysaccharide, humic acid or other humic substances.

### Anti-parasitic Antibodies

25. In another aspect, the pathogen can be a parasite and the antigen a parasitic antigen. Disclosed herein are antibodies and compositions comprising said antibodies, such as, for example, vaccines, passive immunotherapy, and passive immunizations wherein the antibody is specific for a parasitic antigen from a parasite selected from the group consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica. For example, the antigen can be parasitophorous vacuole membrane-enclosed merozoite structures, galactose-inhibitable adherence protein, TSOL 16, MSP1, AMA1, Tryptophan rich antigens, MIC1, MAG1, or SAG1.

### Anti-fungal Antibodies

26. Also disclosed, the pathogen can be a fungus and the antigen a fungal antigen. Disclosed herein are antibodies and compositions comprising said antibodies, such as, for example, vaccines, passive immunotherapy, and passive immunizations wherein the antibody is specific for a fungal antigen from a fungielected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata. For example, the fungal antigen can be Dse1, Int1, glucuronoxylomannan capsular polysaccharide, mannose polymers (mannan), galactomannan, Asp f 16 and Asp f 9, O-glycosylhydroases, β-endoglucanases, CRH-like proteins, Enolase, pyruvate decarboxylase, aldolase, pyruvate carboxylase, transketolase, phosphoglucomutase, HSP 30, 60, 80 and 90, AHP1, Elongation factor 1, Leishmanial elongation factor 4a, Phosphoglucomutase, Ribosomal L10 protein, PEP2, formate dehydrogenase, Histone H3, or Chitin.

### Antibodies to Antigens Present on Pathogens at Mucosal Surfaces

Many of the viral, bacterial, fungal, and parasitic infections to which the disclosed antibodies are raised are infections of mucosal surfaces. Typically, mucosal antibody provides a primary line of defense against pathogen invasion. The current dogma for antibody-mediated mucosal immunity is that polymeric IgA receptor (pIgR)-mediated transcytosis of dimeric IgA (dIgA) crosses epithelial barrier and releases secretory IgA (S-IgA) into mucosal secretions. For many years, IgA has been considered as a major antibody in seeding mucosal immunity. The role of IgG in mucosal immunity has been largely neglected although IgG is a major dominant isotype in the lung. Intriguingly, acidic endosomes appear to be the primary compartment in which FcRn resides and functions, and endocytosed virions initiate fusion of their envelopes within these compartments. Therefore, the endosome is an ideal site for the transcytosed IgG to meet internalized virions within polarized epithelial cells. Thus, FcRn traffics extracellular virus-specific IgG to the endosomes of epithelial cells, where it prevents virus replication. To show this, an mAb, Y8-10C2 (Y8), traditionally considered to be “non-neutralizing” IgG, is in fact capable of blocking viral infection in polarized epithelial cells via a mechanism which is dependent on FcRn-mediated IgG transport. It is intriguing that Y8 mAb binds to the globular but not the fusion domain of the stalk region of influenza HA. By binding to low pH-induced monomeric HA molecules, Y8 mAb prevented a structural transition of HA required for membrane fusion. Thus, Y8 mAb prevents viral membrane fusion and the subsequent entry of viral contents into the cytosol, finally resulting in the transport of virions to the lysosome for destruction.

27. In one aspect, disclosed herein are antibodies specific for antigens present on pathogens at mucosal surfaces including non-neutralizing antibodies, wherein the isotype is changed from IgA to IgG.

### Anti-cancer Antibodies

28. It is understood and herein contemplated that the antibodies disclosed herein can also be useful in treating and or diagnosing a cancer. Thus, disclosed herein are antibodies specific for a non-surface expressed antigen or an antigenic determinant that is only accessible to an antibody through a conformational change of the antigen wherein the antigen is encoded by a cancer. Accordingly, in one aspect, disclosed herein are antibodies specific for a non-surface expressed antigen or an antigenic determinant that is only accessible to an antibody through a conformational change of the antigen wherein the antigen is encoded by a cancer and the cancer is selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer. It is understood and herein contemplated that the cancer antigen can be a oncogenic protein. Furthermore, it is contemplated herein that the cancer antigen to which the dislosed antibody is specific can be a growth factor or mitogen, including but not limited to c-S is, PDGF, CSF-1, EGF, PMA, IGF-1, IGF-2, IL-1, IL-2, IL-6, IL-8, estrogens, androgens, VEGF or FGF. Alternatively, the disclosed antibodies can be specific for a tyrosine kinase, including but not limited to Src-family proteins, Syk-ZAP-70, BTK, pp 125, E6 and E7 from Human papillomavirus, or JAK family proteins or a serine/threonine kinase, including but not limited to Raf, cyclin-dependent kinases, protein kinase A (PKA), protein kinase B (AKT), protein kinase C(PKC), phosphatidylinositol 3-kinase (PI3K), mTOR, mitogen-activated protein kinases (MAPKs), ERK1, ERK2, ERK3, ERK4, ERK5, ERK6, ERK7, JNKs, p38, MKK1, MKK2, RSK kinase, ASK1, TAK1, MLK3, TAOK1, Ca2+/calmodulin-dependent protein kinases (CaM Kinase), ribosomal S6 kinase or IRAK1. In another aspect, the disclosed antibodies can be specific for a regulatory GTPase, including but not limited to Ras, Rho, Rab, Arf, Ran, Ral, or Rac or a transcription factor, including but not limited to myc or c-Myc, a STAT family protein, a HOX family protein, NF-KB, AP-1, SP1, NF-1, Oct-1, ATF/CREB, C/EBP, Elk-1, c-Jun, c-Fos or steroid recpetors. It is further contemplated herein that the disclosed antibodies can be specific for an antigen that is a protein target that has been pathologically phosphorylated or dephosphorylated. For example, when AKT is phosphorylated it is activated. When it is constitutively phosphorylated it can result in hyperproliferation and cancer. Therefore, phosphor-AKT is an example of one such antigen. Likewise, hyperpohsphoylated retinoblastoma protein (Rb) is a useful antigen to target in order to decrease proliferation in cancer. Likewise, phosphorylation of intercellular tyrosines of receptor tyrosine kinases, like EGFR, FGFR, and VEGFR, results in the activation of signal transduction, the net result of which often has a bearing on survival and proliferation of the cell. This phosphorylation site is also an adequate antigen target. Conversely, peptidyl-prolyl cis/trans isomerase (Pin 1) has been implicated in multiple types of cancer and is oncogenic when it is hypophosphorylated. Thus, hypophosphorylated Pin1 is also a useful antigen.

### Antibodies to Allergens

29. In addition to antibodies disclosed herein that are specific for pathogenic antigens or cancer antigens, it is contemplated herein that the disclosed antibodies can be specific for an allergen. such antibodies are useful in passive immunotherapies and passive immunizations, for example, in sensitization therapy, as a mechanism for stifling an allergic response, or Rh incompatibility. Accordingly, in one aspect, disclosed herein are antibodies specific for a non-surface expressed antigen or an antigenic determinant that is only accessible to an antibody through a conformational change of the antigen wherein the antigen is an allergen selected from the allergens from group consisting of house Mites Mite, House Dust Dermatophagoides farinae Mite, House Dust Dermatophagoides pteronyssinus Mite, Acarus siro Food/Storage Mite, House Dust Blomia tropicalis Mite, Storage Chortoglyphus arcuates Mite, House Dust Euroglyphus maynei Mite, Lepidoglyphus Food/Storage destructor Mite, Tyrophagus Food/Storage putrescentiae Mite, House Dust Glycyphagus domesticus Venoms Bumble Bee Bombus spp. Venom European Hornet Vespa crabro Venom Honey Bee Apis mellifera. Venom Mixed Hornet Dolichovespula Venom spp Mixed Paper Polistes spp. Wasp Venom Mixed Yellow Vespula spp. Jacket Venom White (bald)-Dolichovespula faced Hornet maculate Venom Yellow Hornet Dolichovespula Venom arenaria Insects Ant, Carpenter Camponotus pennsylvanicus Ant, Fire Solenopsis invicta Ant, Fire Solenopsis richteri Cockroach, Periplaneta American Americana Cockroach, Blattella German germanica Cockroach, Blatta orientalis Oriental Horse Fly Tabanus spp. House Fly Musca domestica Mayfly Ephemeroptera spp. Mosquito Culicidae sp. Moth Heterocera spp. Epithelia, Dander, Hair & Feathers Canary Feathers Serinus canaria Cat Epithelia Felis catus (domesticus) Cattle Epithelia Bos Taurus Chicken Feathers Gallus gallus (domesticus) Dog Epithella, Canis familiaris Mixed Breeds Duck Feathers Anas platyrhynchos Gerbil Epithelia Meriones unguiculatus Goat Epithelia Capra hircus Goose Feathers Anser domesticus Guinea Pig Cavia porcellus Epithelia (cobaya) Hamster Epithelia Mesocricetus auratus Hog Epithelia Sus scrofa Horse Epithelia Equus caballus Mouse Epithelia Mus musculus Parakeet Feathers Psittacidae spp. Pigeon Feathers Columba fasciata Rabbit Epithelia Oryctolagus cuniculus Rat Spithelia Rettus norvegicus Wool, Sheep Ovis aries Dander Cat Felis catus dander/Antigen (domesticus) Dog Dander, Canis familiaris Mixed-Breed Poodle Dander Canis familiaris Fungi Acremonium Cephalosporium strictum acremonium Alternaria Alternaria alternate tenuis Aspergillus Aspergillus amstelodami glaucus Aspergillus flavus Aspergillus furmigatus Aspergillus nidulans Aspergillus niger Aspergillus terreus Aspergillus versicolor Aureobasidium Pullularia pullulans pullulans Bipolaris Drechslera sorokiniana sorokiniana, Helminthosporium sativum Botrytis cinerea Candida albicans Chaetomium globosum Cladosporium herbarum Cladosporium Hormodendrum sphaerospermum hordei Drechslere Curvularia spicifera spicifera Epicoccum Epicoccum nigrum purpurascens Epidermophyton floccosum Fusarium moniliforme Fusarium solani Geotrichum Oospora lactis candidum Gliocladium Gliocladium viride deliquescens Helminthosporium Spondylocladium solani atrovirens Microsporum Microsporum canis lanosum Mucor Mucor mucedo circinelloides f. circinelloides Mucor Mucor circinelloides f. racemosus lusitanicus Mucor plumbeus Mycogone perniciosa Neurospora Neurospora intermedia sitophila, Monilia sitophila Nigrospora oryzae Paecilomyces variotii Penicillium brevi-compactum Penicillium camembertii Penicillium chrysogenum Penicillium digitatum Penicillium expensum Penicillium notatum Penicillium roquefortii Phoma betae Phomma Phoma herbarum pigmentivora Rhigopus oryzae Rhizopus arrhizus Rhizopus Rhizopus stolonifer nigricans Rhodotorula Rhodotorula mucilaginosa rubra var. mucilaginosa Saccharomyces cerevisiae Scopulariopsis brevicaulis Serpula lacrymans Merulius lacrymans Setosphaeria Exserohilum rostrata rostratum, Helminthosporium halodes Stemphylium botryosum Stemphylium solani Trichoderma Trichoderma harzianum viride Trichophyton Trichophyton mentagrophytes interdigitale Trichophyton rubrum Trichothecium Cephalothecium roseum roseum Smuts Barley Smut Ustilago nuda Bermuda Grass ustilago Smut cynodontis Corn Smut Ustilago maydis Johnson Grass Sporisorium Smut cruentum Oat Smut Ustilago avenae Wheat Smut Ustilago tritici Grass Pollens Bahia Paspalum notatum Bermuda Cynodon dactylon Blue, Canada Poa compressa Brome, Smooth Bromus inermis Canary Phalaris arundinacea Corn Zea mays Couch/Quack Elytrigia repens (Agropyron repens) Johnson Sorghum, halepense Kentucky Blue Poa pratensis Meadow Fescue Festuca pratensis (elatior) Oat, Cultivated Avena sativa Orchard Dactylis glomerata Red Top Agrostis gigantean (alba) Rye, Cultivated Secale cereale Rye, Giant Wild Leymus (Elymus) condensatus Rye, Italian Lolium perenne ssp. multiflorum Rye, Perennial Lolium perenne Sweet Vernal Anthoxanehum odoratum Timothy Phleum pratense Velvet Holcus lanatus Wheat, Cultivated Triticum aestivum Wheatgrass, Elymus Western (Agropyron) smithii Weed Pollens Allscale Atriplex polycarpa Baccharis Baccharis halimifolia Baccharis Baccharis sarothroides Burrobrush Hymenoclea salsola Careless Weed Amaranthus hybridus Cocklebur Xanthium strumarium (commune) Dock, Yellow Rumex crispus Dog Fennel Eupatorium capillifolium Goldenrod Solidago spp. Hemp, Western Amaranthus Water tuberculatus (Acnida tamariscina) Iodine Bush Allenrolfea occidentalis Jerusalem Oak Chenopodium botrys Kochia/Firebush Kochia scoparia Lambs Quarter Chenopodium album Marsh Elder, Iva xanthifolia Burweed Marsh Elder, Iva angustifolia Narrowleaf Marsh Elder, Iva annua Rough (ciliata) Mexican Tea Chenopodium ambrosioides Mugwort, Artemisia Common vulgaris Mugwort, Artemisia Darkleaved ludoviciana Nettle Urtica dioica Palmer's Amaranthus Amaranth palmeri Pigweed, Amaranthus Redroot/Rough retroflexus Pigweed, Spiny Amaranthus spinosus Plantain, English Plantago lanceolata Poverty Weed Iva axillaris Quailbrush Atriplex lentiformis Rabbit Bush Ambrosia deltoidea Ragweed, Desert Ambrosia dumosa Ragweed, False Ambrosia acanthicarpa Ragweed, Giant Ambrosia trifida Ragweed, Short Ambrosia artemisiifolia Ragweed, Slender Ambrosia confertiflora Ragweed, Ambrosia Southern bidentata Ragweed, Ambrosia Western psilostachya Russian Thistle Salsola kali (pestifer) Sage, Coastal Artemisia californica Sage, Pasture Artemisia frigida Sagebrush, Artemisia Common tridentate Saltbush, Annual Atriplex wrightii Shadscale Atriplex confertifolia Sorrel, Red/Sheep Rumex acetosella Wingscale Atriplex canescens Wormwood, Artemisia annua Annual Tree Pollens Acacia Acacia spp. Alder, European Alnus glutinosa Alder, Red Alnus rubra Alder, Tag Alnus incana ssp. rugosa Alder, White Alnus rhombifolia Ash, Arizona Fraxinus velutina Ash, Green/Red Fraxinus pennsylvanica Ash, Oregon Fraxinus latifolia Ash, White Fraxinus americana Aspen Populus tremuloides Bayberry Myrica cerifera Beech, American Fagus grandifolia (americana) Beefwood/Austral Casuarina ian Pine equisetifolia Birch, Betula lenta Black/Sweet Birch, European Betula pendula White Birch, Red/River Betula nigra Birch, Spring Betula occidentalis (fontinalis) Birch, White Betula populifolia Box Elder Acer negundo Cedar, Japanese Cryptomeria japonica Cedar, Mountain Juniperus ashei (sabinoides) Cedar, Red Juniperus virginiana Cedar, Salt Tamarix gallica Cottonwood, Populus Black balsamifera ssp. trichocarpa Cottonwood, Populus Eastern deltoides Cottonwood, Populus Fremont fremontii Cottonwood, Rio Populus Grande wislizeni Cottonwood, Populus Western monilifera (sargentii) Cypress, Arizona Cupressus arizonica Cypress, Bald Taxodium distichum Cypress, Italian Cupressus sempervirens Elm, American Ulmus americana Elm, Cedar Ulmus crassifolia Elm, Siberian Ulmus pumila Eucalyptus Eucalyptus globulus Hackberry Celtis occidentalis Hazelnut Corylus americana Hazelnut, Corylus European avellana Hickory, Pignut Carya glabra Hickory, Carya ovata Shagbark Hickory, Carya laciniosa Shellbark Hickory, White Carya alba Juniper, Oneseed Juniperus monosperma Juniper, Pinchot Juniperus pinchotii Juniper, Rocky Juniperus Mountain scopulorum Juniper, Utah Juniperus osteosperma Juniper, Western Juniperus occidentalis Locust Blossom, Robinia Black pseudoacacia Mango Blossom Mangifera indica Maple, Coast Acer macrophyllum Maple, Red Acer rubrum Maple, Silver Acer saccharinum Maple, Sugar Acer saccharum Melaleuca Melaleuca quinquenervia (leucadendron) Mesquite Prosopis glandulosa (julifiora) Mulberry, Paper Broussonetia papyrifera Mulberry, Red Morus rubra Mulberry, White Morus alba Oak, Quercus Arizona/Gambel gambeiji Oak, Black Quercus velutina, Oak, Bur Quercus macrocarpa Oak, California Quercus Black kelloggii Oak, California Quercus Live agrifolia Oak, California Quercus lobata White/Valley Oak, English Quercus robur Oak, Holly Quercus ilex Oak, Post Quercus stellata Oak, Red Quercus rubra Oak, Scrub Quercus dumosa Oak, Virginia Quercus Live virginiana Oak, Water Quercus nigra Oak, Western Quercus White/Gany garryana Oak, White Quercus alba Olive Olea europaea Olive, Russian Elaeagnus angustifolia Orange Pollen Citrus sinensis Palm, Queen Arecastrum romanzoffianum (Cocos plumosa) Pecan Carya illinoensis Pepper Tree Schinus molle Pepper Schinus Tree/Florida terebinthifolius Holly Pine, Loblolly Pinus taeda Pine, Eastern Pinus strobus White Pine, Longleaf Pinus palustris Pine, Ponderosa Pinus ponderosa Pine, Slash Pinus elliottii Pine, Virginia Pinus virginiana Pine, Western Pinus monticola White Pine, Yellow Pinus echinata Poplar, Lombardy Populus nigra Poplar, White Populus alba Privet Ligustrum vulgare Sweet Gum Liquidambar styraciflua Sycamore, Platanus Eastern occidentalis Sycamore, Platanus Oriental orientalis Sycamore, Platanus Western racemosa Sycamore/London Platanus Plane acerifolia Walnut, Black Juglans nigra Walnut, Juglans California Black californica Walnut, English Juglans regia Willow, Arroyo Salix lasiolepis Willow, Black Salix nigra Willow, Pussy Salix discolor Flowers: Wild & Cultivated Daisy, Ox-Eye Chrysanthemum leucanthemum Dandelion Taraxacum officinale Sunflower Helianthus annuus Cultivated Farm Plant Pollens Alfalfa Medicago sativa Castor Bean Ricinus communis Clover, Red Trifolium pratense Mustard Brassica spp. Sugar Beet Beta vulgaris Plant Food Almond Prunus dulcis Apple Malus pumila Apricot Prunus armeniaca Banana Musa paradisiaca (sapientum) Barley Hordeum vulgare Bean, Lima Phaseolus lunatus Bean, Navy Phaseolus vulgaris Bean, Pinto Phaseolus sp. Bean, Red Kidney Phaseolus sp. Bean, Phaseolus String/Green vulgaris Blackberry Rubus allegheniensis Blueberry Vaccinium sp. Broccoli Brassica oleracea var. botrytis Buckwheat Fagopyrum esculentum Cabbage Brassica oleracea var. capitata Cacao Bean Theobroma cacao Cantaloupe Cucumis melo Carrot Daucus carota Cauliflower Brassica oleracea var. botrytis Celery Apium graveolens var. dulce Cherry Prunus sp. Cinnamon Cinnamomum verum Coffee Coffee arabica Corn Zea mays Cranberry Vaccinium macrocarpon Cucumber Cucumis sativus Garlic Allium sativum Ginger Zingiber officinale Grape Vitis sp. Grapefruit Citrus paradisi Hops Humulus lupulus Lemon Citrus limon Lettuce Lactuca sativa Malt Mushroom Agaricus campestris Mustard Brassica sp. Nutmeg Myristica fragrans Oat Avena sativa Olive, Green Olea europaea Onion Allium cepa var. cepa Orange Citrus sinensis Pea, Blackeye Vigna unguiculata Pea, Green Pisum sativum (English) Peach Prunus persica Pear Pyrus communis Pepper, Black Piper nigrum Pepper, Green Capsicum annuum var. annuum Pineapple Ananas comosus Potato, Sweet Ipomoea batatas Potato, White Solanum tuberosum Raspberry Rubus idaeus var. idaeus Rice Oryza sativa Rye Secale cereale Sesame Seed Sesamum orientale (indicum) Soybean Glycine max Spinach Spinacia oleracea Squash, Yellow Cucurbita pepo var. melopepo Strawberry Fragaria chiloensis Tomato Lycopersicon esculentum (lycopersicum) Turnip Brassica rapa var. rapa Vanilla Bean Vanilla planifolia Watermelon Citrullus lanatus var. lanatus Wheat, Whole Triticum aestivum Fish & Shellfish Bass, Black Micropterus sp. Catfish Ictalurus punctatus Clam Mercenaria mercenaria Codfish Gadus morhua Crab Callinectes sapidus Flounder Platichthys sp. Halibut Hippoglossus sp. Lobster Homarus americanus Mackerel Scomber scombrus Oyster Crassostrea virginica Perch Sebastes marinus Salmon Salmo salar Sardine Clupeiformes Scallop Pectan magellanicus Shrimp Penaeus sp. Trout, Lake Salvelinus sp. Tuna Fish Thunnus sp Animal Foods Beef Bos taurus Lamb Ovis aries Pork Sus scrofa Poultry Products Chicken Gallus gallus Egg, Chicken, Gallus gallus. White Egg (Gallus gallus), Yolk (Meleagris gallopavo), Casein, Brazil Nut Bertholletia excels, Cashew Nut Anacardium occidentale, Coconut Cocos nucifera, Filbert/Hazelnut Corylus Americana, Peanut Arachis hypogaea, Pecan Carya illinoensis, Walnut, Black Juglans nigra Walnut, English Juglans regia, and latex.

### Antibodies to Toxins

1. It is understood and herein contemplated that the antibodies disclosed herein can bind to antigens that are associated with a toxin. Thus, in one aspect, disclosed herein are antibodies specific for an antigen that is present in or on the surface of a toxin (such as an antigenic determinant on the toxin that is only accessible to an antibody through a conformational change of the antigen) or encoded by a toxin. Such antigens include but are not limited to Abrin, Conotoxins Diacetoxyscirpenol Bovine spongiform encephalopathy agent, Ricin, Saxitoxin, Tetrodotoxin, epsilon toxin, Botulinum neurotoxins, Shigatoxin, Staphylococcal enterotoxins, T-2 toxin, Diphtheria toxin, Tetanus toxoid, and pertussis toxin.

1. Antibodies

(1) Antibodies Generally

2. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with viral, bacterial, fungal, or parasitic antigens such that viral, bacterial, fungal, or parasitic infection, replication, or survival is inhibited; the ability to interact with cancer antigens such that metastasis or cancer progression is inhibited; or the ability to interact with allergens. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

3. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

4. Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises one of the viral, bacterial, parasitic, or fungal antigens; one of the cancer antigens, or one of the allergens disclosed herein. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of one of the viral, bacterial, parasitic, or fungal antigens; one of the cancer antigens, or one of the allergens disclosed herein expressed as a fusion protein with human IgG is injected into the host animal.

5. An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing domains of an antibody as fusion proteins. The antigen is produced by inserting a gene fragment in-frame between the signal sequence and the mature protein domain of the antibody nucleotide sequence. This results in the display of the foreign proteins on the surface of the virion. This method allows immunization with whole virus, eliminating the need for purification of target antigens.

6. Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103) Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against one of the viral, bacterial, parasitic, or fungal antigens; one of the cancer antigens, or one of the allergens disclosed herein. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane Antibodies, A Laboratory Manul Cold Spring Harbor Publications, New York, (1988).

7. After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal

8. The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

9. The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for one of the viral, bacterial, parasitic, or fungal antigens; one of the cancer antigens, or one of the allergens disclosed herein and another antigen-combining site having specificity for a different antigen.

10. In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

11. The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

12. An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

13. One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

14. For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

15. Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

16. As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

17. The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

18. Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

19. To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

20. Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

21. Administration of the antibodies can be done as disclosed herein. Nucleic acid approaches for antibody delivery also exist. The broadly neutralizing anti-viral, anti-bacterial, anti-parasitic, anti-fungal, anti-cancer, or anti-allergens disclosed herein antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### Further Compositions

It is understood that the antibodies disclosed herein can be administered alone or as single active ingredient in a composition. It is further contemplated herein that the disclosed antibodies may be administered in a composition comprising one or more additional active ingredients. For example, disclosed herein are compositions comprising the antibodies disclosed herein and one or more T cell determinants and/or one or more antibodies to extracellular antigens.

2. Pharmaceutical Carriers/Delivery of Pharamceutical Products

22. As described above, the disclosed antibodies can be administered directly or as part of a larger composition. In addition to the disclosed antibodies, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

23. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

24. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

25. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

26. The compositions, including antibodies, can be used therapeutically in combination with a pharmaceutically acceptable carrier or excipient.

27. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

28. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

29. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

30. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

31. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

32. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

33. Compositions for oral administration include powders or granules, suspensions or solutions in water or non-aqueous media, capsules, sachets, or tablets. Thickeners, flavorings, diluents, emulsifiers, dispersing aids or binders may be desirable.

34. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

35. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

36. Following administration of a disclosed composition, such as the antibodies disclosed herein, for treating, inhibiting, or preventing a viral infection, bacterial infection, fungal infection, parasitic infection, cancer, or an allergic reaction, the efficacy of the therapeutic antibody can be assessed in various ways well known to the skilled practitioner. For instance, one of ordinary skill in the art will understand that a composition, such as an antibody, disclosed herein is efficacious in treating or inhibiting an influenza infection in a subject by observing that the composition reduces viral load or prevents a further increase in viral load.

37. The compositions that inhibit viral infection, bacterial infection, fungal infection, parasitic infection, cancer, or an allergic reactions disclosed herein may be administered prophylactically to patients or subjects who are at risk for viral, bacterial, fungal, or parasitic exposure; cancer, an allergic reaction, or exposure to a toxin.

3. Nucleic Acids

38. There are a variety of molecules disclosed herein that are nucleic acid based, including for example the nucleic acids that encode, for example the antibodies disclosed herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

39. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

40. A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

41. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

42. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

43. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

44. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Functional Nucleic Acids

45. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

46. Functional nucleic acid molecules can interact with any macromolecule, such as DNA, RNA, polypeptides, or carbohydrate chains. Thus, functional nucleic acids can interact with the mRNA of any of the disclosed nucleic acids, such as viral polymerases, integrase, reverse transcriptases, glycoproteins, or capsid proteins; bacterial cell wall proteins and the like disclosed herein, and oncoggenes. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

47. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation.

4. Antioxidants

48. Generally, antioxidants are compounds that get react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The compositions can contain any antioxidants, and a non-limiting list would included but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly .alpha.-tocopherol, etc., vitamin E succinate, and trolox (a soluble Vitamin E analog) Vitamin C (ascoribic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39: 301, 1988 and Machlin L. J. and Bendich, F.A.S.E.B. Journal Vol. 1:441-445, 1987 which are incorporated herein by reference for material related to antioxidants.

49. Flavonoids, also known as “phenylchromones,” are naturally occurring, water-soluble compounds which have antioxidant characteristics. Flavonoids are widely distributed in vascular plants and are found in numerous vegetables, fruits and beverages such as tea and wine (particularly red wine). Flavonoids are conjugated aromatic compounds. The most widely occurring flavonoids are flavones and flavonols (for example, myricetin, (3,5,7,3′,4′,5′,-hexahydroxyflavone), quercetin (3,5,7,3′,4′-pentahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof and quercetin).

5. Nucleic Acid Delivery

50. In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada) or retroviral vector. Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

51. As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

52. As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

53. Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

54. As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

55. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

C. Methods of Treating or Inhibiting Disease

56. The antibodies and compositions disclosed herein can be used, for example, to bind antigens or antigenic determinants typically not available to naturally occurring antibodies and provide a therapeutic or prophylactic benefit to a subject receiving the antibody or compositions. Thus, in one aspect, disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen. The antibodies can be neutralizing or non-neutralizing antibodies.

57. Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Similarly, a disclosed method of treating or inhibiting a pathogenic infection is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

58. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

59. It is understood and herein contemplated that the disclosed methods can be used to treat or inhibit any disease or conditions such as a pathogenic infection (e.g., viral infection, bacterial infection, fungal infection, parasitic infection), cancer, or an allergic reaction.

60. In one aspect, disclosed herein are methods of treatment or inhibition wherein the pathogenic infection is a viral infection. It is understood that the viral infection can be any viral infection for which an antibody has been raised and administered to the subject. Thus, for example, disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the disease is a viral infection selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A (including H1N1 or other Swine H1), Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In another aspect, disclosed herein are methods of treating and inhibiting a viral infection wherein the antigen is a viral glycoprotein (GP), portal protein, tegument protein, capsid protein, DNA polymerase, RNA polymerase, reverse transcriptase, protease, integrase, DNA-binding protein, nucleoprotein (NP), nuclear matric protein, envelope protein (ENV), nuclear antigen, membrane protein, proteins encoded by viral early genes, group specific antigen (gag) protein, hemagglutinin (HA), neuraminidase (NA), or matrix protein. Specific examples of viral antigens include but are not limited to ENV, GP160 (HIV) GP120 (HIV), GP41 (HIV), EBNA-1, EBNA-2, EBNA-3, LMP-1, LMP-2, E1, E2, E3, E4, E5, E6, E7, NSP1, NSP2, NSP3, NSP4, NSP5, NSP10, NSP14, NSP15, NSP16, NSP29, G35P, G38P, G39P, zygocin protein, VP5 protein, 3AB protein, L4-22K protein, L4-100K protein, ORF 17 protein, S7 protein, S9 protein, S10 protein, HBXIP protein, UL3.5 protein, virus-infected-associated antigen protein, 3ABC protein, Cng protein, 2 BC protein, p58 protein, A40R protein, vpu protein, VPX protein, BPLF1 protein, NEF protein, SGTA protein, UL102 protein, p121 protein, VP35 protein, SPP1 Pac region protein, pX protein, N protein, agnoprotein, sigma NS protein, phage repressor proteins, U(S)3 protein kinase, ToxR protein, LexA protein, lambda CI repressor protein, Mu Ner protein, and Tat proteins.

61. In one aspect, disclosed herein are methods of treatment or inhibition wherein the pathogenic infection is a bacterial infection. It is understood that the viral infection can be any bacterial infection for which an antibody has been raised and administered to the subject. Thus, for example, disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the disease is a bacterial infection selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In one aspect, the antigens against which antibodies are raised in the disclosed methods can be a bacterial surface protein including but not limited to bacterial oligosaccharide, polysaccharide, or lipopolysaccharide; a protein associated with fimbrial structure and biogenesis, antimicrobial resistance, heavy metal transport, bacterial adhesion, extracytoplasmic substrate trafficking, or secreted hydrolases; exopolysaccharide; humic acid; N-acetylmuramic acid (NAM); N-acetylglucosamine (NAG); teichoic acids including ribitol teichoic acid and glycerol teichoic acid; O-antigen; Lipid A; pilin proteins; Porin; MA0829; or SbsB. In yet another aspect, the antigen can be a a component of a microbial biofilm, examples of which include but are not limited to exopolysaccharide, humic acid or other humic substances.

62. In another apect, the disclosed methods can be used to treat or inhibit a parasitic infection. Thus, for example, disclosed herein are methods wherein the disease is a parasitic infection, and wherein the parasitic infection is an infection with a parasite selected from the group consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica. It is understood and herein contemplated that the disclosed methods of inhibiting or treating a parasitic infection can comprise administering an antibody to a parasitic antigen including but not limited to parasitophorous vacuole membrane-enclosed merozoite structures, galactose-inhibitable adherence protein, TSOL 16, MSP1, AMA1, Tryptophan rich antigens, MIC1, MAG1, and SAG1.

63. In another apect, the disclosed methods can be used to treat or inhibit a fungal infection. Thus disclosed herein are disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the disease is a fungal infection, and wherein the fungal infection is an infection with a fungi selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata. In one aspect, the fungal antigens against which antibodies are raised in the disclosed methods can be Dse1, Int1, glucuronoxylomannan capsular polysaccharide, mannose polymers (mannan), galactomannan, Asp f 16 and Asp f 9, O-glycosylhydroases, β-endoglucanases, CRH-like proteins, Enolase, pyruvate decarboxylase, aldolase, pyruvate carboxylase, transketolase, phosphoglucomutase, HSP 30, 60, 80 and 90, AHP1, Elongation factor 1, Leishmanial elongation factor 4a, Phosphoglucomutase, Ribosomal L10 protein, PEP2, formate dehydrogenase, Histone H3, or Chitin.

64. The principals governing the disclosed methods of treating or inhibiting pathogenic infections are equally applicable to treat any disease where uncontrolled cellular proliferation occurs such as cancers. Thus, in one aspect, disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the disease is a cancer selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer. In one aspect, the disclosed antibodies for use in the methods described herein can be directed to cancer antigens including but not limited to c-Sis, PDGF, CSF-1, EGF, PMA, IGF-1, IGF-2, IL-1, IL-2, IL-6, IL-8, estrogens, androgens, VEGF, FGF, Src-family proteins, Syk-ZAP-70, BTK, pp 125, E6 and E7 from Human papillomavirus, JAK family proteins, Raf, cyclin-dependent kinases, protein kinase A (PKA), protein kinase B (AKT), protein kinase C(PKC), phosphatidylinositol 3-kinase (PI3K), mTOR, mitogen-activated protein kinases (MAPKs), ERK1, ERK2, ERK3, ERK4, ERK5, ERK6, ERK7, JNKs, p38, MKK1, MKK2, RSK kinase, ASK1, TAK1, MLK3, TAOK1, Ca2+/calmodulin-dependent protein kinases (CaM Kinase), ribosomal S6 kinase, IRAK1, Ras, Rho, Rab, Arf, Ran, Ral, Rac, myc or c-Myc, a STAT family protein, a HOX family protein, NF-κB, AP-1, SP1, NF-1, Oct-1, ATF/CREB, C/EBP, Elk-1, c-Jun, c-Fos or steroid recpetors.

65. In addition to the disclosed method of treating pathogenic diseases and cancers, the antibodies disclosed herein can also be used to inhibit or treat (i.e., reduce) an allergic reaction. Thus disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the condition is an allergic reaction to an antigen selected from the allergens from group consisting of house Mites Mite, House Dust Dermatophagoides farinae Mite, House Dust Dermatophagoides pteronyssinus Mite, Acarus siro Food/Storage Mite, House Dust Blomia tropicalis Mite, Storage Chortoglyphus arcuates Mite, House Dust Euroglyphus maynei Mite, Lepidoglyphus Food/Storage destructor Mite, Tyrophagus Food/Storage putrescentiae Mite, House Dust Glycyphagus domesticus Venoms Bumble Bee Bombus spp. Venom European Hornet Vespa crabro Venom Honey Bee Apis mellifera. Venom Mixed Hornet Dolichovespula Venom spp Mixed Paper Polistes spp. Wasp Venom Mixed Yellow Vespula spp. Jacket Venom White (bald)-Dolichovespula faced Hornet maculate Venom Yellow Hornet Dolichovespula Venom arenaria Insects Ant, Carpenter Camponotus pennsylvanicus Ant, Fire Solenopsis invicta Ant, Fire Solenopsis richteri Cockroach, Periplaneta American Americana Cockroach, Blattella German germanica Cockroach, Blatta orientalis Oriental Horse Fly Tabanus spp. House Fly Musca domestica Mayfly Ephemeroptera spp. Mosquito Culicidae sp. Moth Heterocera spp. Epithelia, Dander, Hair & Feathers Canary Feathers Serinus canaria Cat Epithelia Felis catus (domesticus) Cattle Epithelia Bos Taurus Chicken Feathers Gallus gallus (domesticus) Dog Epithella, Canis familiaris Mixed Breeds Duck Feathers Anas platyrhynchos Gerbil Epithelia Meriones unguiculatus Goat Epithelia Capra hircus Goose Feathers Anser domesticus Guinea Pig Cavia porcellus Epithelia (cobaya) Hamster Epithelia Mesocricetus auratus Hog Epithelia Sus scrofa Horse Epithelia Equus caballus Mouse Epithelia Mus musculus Parakeet Feathers Psittacidae spp. Pigeon Feathers Columba fasciata Rabbit Epithelia Oryctolagus cuniculus Rat Spithelia Rettus norvegicus Wool, Sheep Ovis aries Dander Cat Felis catus dander/Antigen (domesticus) Dog Dander, Canis familiaris Mixed-Breed Poodle Dander Canis familiaris Fungi Acremonium Cephalosporium strictum acremonium Alternaria Alternaria alternate tenuis Aspergillus Aspergillus amstelodami glaucus Aspergillus flavus Aspergillus furmigatus Aspergillus nidulans Aspergillus niger Aspergillus terreus Aspergillus versicolor Aureobasidium Pullularia pullulans pullulans Bipolaris Drechslera sorokiniana sorokiniana, Helminthosporium sativum Botrytis cinerea Candida albicans Chaetomium globosum Cladosporium herbarum Cladosporium Hormodendrum sphaerospermum hordei Drechslere Curvularia spicifera spicifera Epicoccum Epicoccum nigrum purpurascens Epidermophyton floccosum Fusarium moniliforme Fusarium solani Geotrichum Oospora lactis candidum Gliocladium Gliocladium viride deliquescens Helminthosporium Spondylocladium solani atrovirens Microsporum Microsporum canis lanosum Mucor Mucor mucedo circinelloides f. circinelloides Mucor Mucor circinelloides f. racemosus lusitanicus Mucor plumbeus Mycogone perniciosa Neurospora Neurospora intermedia sitophila, Monilia sitophila Nigrospora oryzae Paecilomyces variotii Penicillium brevi-compactum Penicillium camembertii Penicillium chrysogenum Penicillium digitatum Penicillium expensum Penicillium notatum Penicillium roquefortii Phoma betae Phomma Phoma herbarum pigmentivora Rhigopus oryzae Rhizopus arrhizus Rhizopus Rhizopus stolonifer nigricans Rhodotorula Rhodotorula mucilaginosa rubra var. mucilaginosa Saccharomyces cerevisiae Scopulariopsis brevicaulis Serpula lacrymans Merulius lacrymans Setosphaeria Exserohilum rostrata rostratum, Helminthosporium halodes Stemphylium botryosum Stemphylium solani Trichoderma Trichoderma harzianum viride Trichophyton Trichophyton mentagrophytes interdigitale Trichophyton rubrum Trichothecium Cephalothecium roseum roseum Smuts Barley Smut Ustilago nuda Bermuda Grass ustilago Smut cynodontis Corn Smut Ustilago maydis Johnson Grass Sporisorium Smut cruentum Oat Smut Ustilago avenae Wheat Smut Ustilago tritici Grass Pollens Bahia Paspalum notatum Bermuda Cynodon dactylon Blue, Canada Poa compressa Brome, Smooth Bromus inermis Canary Phalaris arundinacea Corn Zea mays Couch/Quack Elytrigia repens (Agropyron repens) Johnson Sorghum, halepense Kentucky Blue Poa pratensis Meadow Fescue Festuca pratensis (elatior) Oat, Cultivated Avena sativa Orchard Dactylis glomerata Red Top Agrostis gigantean (alba) Rye, Cultivated Secale cereale Rye, Giant Wild Leymus (Elymus) condensatus Rye, Italian Lolium perenne ssp. multiflorum Rye, Perennial Lolium perenne Sweet Vernal Anthoxanehum odoratum Timothy Phleum pratense Velvet Holcus lanatus Wheat, Cultivated Triticum aestivum Wheatgrass, Elymus Western (Agropyron) smithii Weed Pollens Allscale Atriplex polycarpa Baccharis Baccharis halimifolia Baccharis Baccharis sarothroides Burrobrush Hymenoclea salsola Careless Weed Amaranthus hybridus Cocklebur Xanthium strumarium (commune) Dock, Yellow Rumex crispus Dog Fennel Eupatorium capillifolium Goldenrod Solidago spp. Hemp, Western Amaranthus Water tuberculatus (Acnida tamariscina) Iodine Bush Allenrolfea occidentalis Jerusalem Oak Chenopodium botrys Kochia/Firebush Kochia scoparia Lambs Quarter Chenopodium album Marsh Elder, Iva xanthifolia Burweed Marsh Elder, Iva angustifolia Narrowleaf Marsh Elder, Iva annua Rough (ciliata) Mexican Tea Chenopodium ambrosioides Mugwort, Artemisia Common vulgaris Mugwort, Artemisia Darkleaved ludoviciana Nettle Urtica dioica Palmer's Amaranthus Amaranth palmeri Pigweed, Amaranthus Redroot/Rough retroflexus Pigweed, Spiny Amaranthus spinosus Plantain, English Plantago lanceolata Poverty Weed Iva axillaris Quailbrush Atriplex lentiformis Rabbit Bush Ambrosia deltoidea Ragweed, Desert Ambrosia dumosa Ragweed, False Ambrosia acanthicarpa Ragweed, Giant Ambrosia trifida Ragweed, Short Ambrosia artemisiifolia Ragweed, Slender Ambrosia confertiflora Ragweed, Ambrosia Southern bidentata Ragweed, Ambrosia Western psilostachya Russian Thistle Salsola kali (pestifer) Sage, Coastal Artemisia californica Sage, Pasture Artemisia frigida Sagebrush, Artemisia Common tridentate Saltbush, Annual Atriplex wrightii Shadscale Atriplex confertifolia Sorrel, Red/Sheep Rumex acetosella Wingscale Atriplex canescens Wormwood, Artemisia annua Annual Tree Pollens Acacia Acacia spp. Alder, European Alnus glutinosa Alder, Red Alnus rubra Alder, Tag Alnus incana ssp. rugosa Alder, White Alnus rhombifolia Ash, Arizona Fraxinus velutina Ash, Green/Red Fraxinus pennsylvanica Ash, Oregon Fraxinus latifolia Ash, White Fraxinus americana Aspen Populus tremuloides Bayberry Myrica cerifera Beech, American Fagus grandifolia (americana) Beefwood/Austral Casuarina ian Pine equisetifolia Birch, Betula lenta Black/Sweet Birch, European Betula pendula White Birch, Red/River Betula nigra Birch, Spring Betula occidentalis (fontinalis) Birch, White Betula populifolia Box Elder Acer negundo Cedar, Japanese Cryptomeria japonica Cedar, Mountain Juniperus ashei (sabinoides) Cedar, Red Juniperus virginiana Cedar, Salt Tamarix gallica Cottonwood, Populus Black balsamifera ssp. trichocarpa Cottonwood, Populus Eastern deltoides Cottonwood, Populus Fremont fremontii Cottonwood, Rio Populus Grande wislizeni Cottonwood, Populus Western monilifera (sargentii) Cypress, Arizona Cupressus arizonica Cypress, Bald Taxodium distichum Cypress, Italian Cupressus sempervirens Elm, American Ulmus americana Elm, Cedar Ulmus crassifolia Elm, Siberian Ulmus pumila Eucalyptus Eucalyptus globulus Hackberry Celtis occidentalis Hazelnut Corylus americana Hazelnut, Corylus European avellana Hickory, Pignut Carya glabra Hickory, Carya ovata Shagbark Hickory, Carya laciniosa Shellbark Hickory, White Carya alba Juniper, Oneseed Juniperus monosperma Juniper, Pinchot Juniperus pinchotii Juniper, Rocky Juniperus Mountain scopulorum Juniper, Utah Juniperus osteosperma Juniper, Western Juniperus occidentalis Locust Blossom, Robinia Black pseudoacacia Mango Blossom Mangifera indica Maple, Coast Acer macrophyllum Maple, Red Acer rubrum Maple, Silver Acer saccharinum Maple, Sugar Acer saccharum Melaleuca Melaleuca quinquenervia (leucadendron) Mesquite Prosopis glandulosa (julifiora) Mulberry, Paper Broussonetia papyrifera Mulberry, Red Morus rubra Mulberry, White Morus alba Oak, Quercus Arizona/Gambel gambeiji Oak, Black Quercus velutina, Oak, Bur Quercus macrocarpa Oak, California Quercus Black kelloggii Oak, California Quercus Live agrifolia Oak, California Quercus lobata White/Valley Oak, English Quercus robur Oak, Holly Quercus ilex Oak, Post Quercus stellata Oak, Red Quercus rubra Oak, Scrub Quercus dumosa Oak, Virginia Quercus Live virginiana Oak, Water Quercus nigra Oak, Western Quercus White/Gany garryana Oak, White Quercus alba Olive Olea europaea Olive, Russian Elaeagnus angustifolia Orange Pollen Citrus sinensis Palm, Queen Arecastrum romanzoffianum (Cocos plumosa) Pecan Carya illinoensis Pepper Tree Schinus molle Pepper Schinus Tree/Florida terebinthifolius Holly Pine, Loblolly Pinus taeda Pine, Eastern Pinus strobus White Pine, Longleaf Pinus palustris Pine, Ponderosa Pinus ponderosa Pine, Slash Pinus elliottii Pine, Virginia Pinus virginiana Pine, Western Pinus monticola White Pine, Yellow Pinus echinata Poplar, Lombardy Populus nigra Poplar, White Populus alba Privet Ligustrum vulgare Sweet Gum Liquidambar styraciflua Sycamore, Platanus Eastern occidentalis Sycamore, Platanus Oriental orientalis Sycamore, Platanus Western racemosa Sycamore/London Platanus Plane acerifolia Walnut, Black Juglans nigra Walnut, Juglans California Black californica Walnut, English Juglans regia Willow, Arroyo Salix lasiolepis Willow, Black Salix nigra Willow, Pussy Salix discolor Flowers: Wild & Cultivated Daisy, Ox-Eye Chrysanthemum leucanthemum Dandelion Taraxacum officinale Sunflower Helianthus annuus Cultivated Farm Plant Pollens Alfalfa Medicago sativa Castor Bean Ricinus communis Clover, Red Trifolium pratense Mustard Brassica spp. Sugar Beet Beta vulgaris Plant Food Almond Prunus dulcis Apple Malus pumila Apricot Prunus armeniaca Banana Musa paradisiaca (sapientum) Barley Hordeum vulgare Bean, Lima Phaseolus lunatus Bean, Navy Phaseolus vulgaris Bean, Pinto Phaseolus sp. Bean, Red Kidney Phaseolus sp. Bean, Phaseolus String/Green vulgaris Blackberry Rubus allegheniensis Blueberry Vaccinium sp. Broccoli Brassica oleracea var. botrytis Buckwheat Fagopyrum esculentum Cabbage Brassica oleracea var. capitata Cacao Bean Theobroma cacao Cantaloupe Cucumis melo Carrot Daucus carota Cauliflower Brassica oleracea var. botrytis Celery Apium graveolens var. dulce Chemy Prunus sp. Cinnamon Cinnamomum verum Coffee Coffee arabica Corn Zea mays Cranberry Vaccinium macrocarpon Cucumber Cucumis sativus Garlic Allium sativum Ginger Zingiber officinale Grape Vitis sp. Grapefruit Citrus paradisi Hops Humulus lupulus Lemon Citrus limon Lettuce Lactuca sativa Malt Mushroom Agaricus campestris Mustard Brassica sp. Nutmeg Myristica fragrans Oat Avena sativa Olive, Green Olea europaea Onion Allium cepa var. cepa Orange Citrus sinensis Pea, Blackeye Vigna unguiculata Pea, Green Pisum sativum (English) Peach Prunus persica Pear Pyrus communis Pepper, Black Piper nigrum Pepper, Green Capsicum annuum var. annuum Pineapple Ananas comosus Potato, Sweet Ipomoea batatas Potato, White Solanum tuberosum Raspberry Rubus idaeus var. idaeus Rice Oryza sativa Rye Secale cereale Sesame Seed Sesamum orientale (indicum) Soybean Glycine max Spinach Spinacia oleracea Squash, Yellow Cucurbita pepo var. melopepo Strawberry Fragaria chiloensis Tomato Lycopersicon esculentum (lycopersicum) Turnip Brassica rapa var. rapa Vanilla Bean Vanilla planifolia Watermelon Citrullus lanatus var. lanatus Wheat, Whole Triticum aestivum Fish & Shellfish Bass, Black Micropterus sp. Catfish Ictalurus punctatus Clam Mercenaria mercenaria Codfish Gadus morhua Crab Callinectes sapidus Flounder Platichthys sp. Halibut Hippoglossus sp. Lobster Homarus americanus Mackerel Scomber scombrus Oyster Crassostrea virginica Perch Sebastes marinus Salmon Salmo salar Sardine Clupeiformes Scallop Pectan magellanicus Shrimp Penaeus sp. Trout, Lake Salvelinus sp. Tuna Fish Thunnus sp Animal Foods Beef Bos taurus Lamb Ovis aries Pork Sus scrofa Poultry Products Chicken Gallus gallus Egg, Chicken, Gallus gallus. White Egg (Gallus gallus), Yolk (Meleagris gallopavo), Casein, Brazil Nut Bertholletia excels, Cashew Nut Anacardium occidentale, Coconut Cocos nucifera, Filbert/Hazelnut Corylus Americana, Peanut Arachis hypogaea, Pecan Carya illinoensis, Walnut, Black Juglans nigra Walnut, English Juglans regia, and latex. Also disclosed are methods further comprising switching isotype of antibody form IgE to IgG.

66. The antibodies disclosed herein can also be used to treat a subject having been exposed to a toxin. Thus, in one aspect, disclosed herein are methods of treating or inhibiting a disease or condition comprising administering to a subject one or more antibodies, wherein each antibody separately specific for a non-surface expressed antigen or an antigenic determinant that is only accessible to an antibody through a conformational change of the antigen, and wherein the antigens include but are not limited to Abrin, Conotoxins Diacetoxyscirpenol Bovine spongiform encephalopathy agent, Ricin, Saxitoxin, Tetrodotoxin, epsilon toxin, Botulinum neurotoxins, Shigatoxin, Staphylococcal enterotoxins, T-2 toxin, Diphtheria toxin, Tetanus toxoid, and pertussis toxin.

D. Methods of Diagnosing or Detecting Exposure

67. It is understood and herein contemplated that one use of the disclosed antibodies is for the diagnosis of a disease or condition or the detection of exposure to an antigen. As the antibodies bind to antigens, the use of a labeled antibody allows for the ability to detect when an antibody has bound a target. In this case, the target can be a viral antigen, bacterial antigen, fungal antigen, parasitic antigen, cancer antigen, allergen, or toxin. The detection of the presence of the labeled antibody would indicate exposure to the target antigen or provide a diagnosis. Thus, disclosed herein are methods of diagnosing a disease or condition in a subject or detecting exposure of a subject to an antigen associated with a disease, condition, or toxin comprising obtaining a tissue sample from the subject and contacting the tissue with one or more antibodies, wherein each antibody separately specific for a non-surface expressed antigen or an antigenic determinant that is only accessible to an antibody through a conformational change of the antigen, wherein the one or more antibodies comprise a detectable label, wherein detection of the one or more antibodies indicates the subject has the disease or condition or indicates exposure to the antigen associated with a disease, condition, or toxin.

68. It is understood and herein contemplated that the tissue sample can include any tissue that can reasonably be extracted from a subject influding, but not limited to blood (including peripheral blood and peripheral blood mononuclear cells), tissue biopsy samples (e.g., spleen, liver, bone marrow, thymus, lung, kidney, brain, salivary glands, skin, lymph nodes, and intestinal tract), and specimens acquired by pulmonary lavage (e.g., bronchoalveolar lavage (BAL)). It is further understood that the disclosed methods can utilize a labeled antibody labeled in any available way the facilitates detection and any method of immunodetection known in the art

69. It is understood that the disclosed diagnostic or detection methods can be used to diagnosis or detect exposure to pathogenic infections (e.g., viral, bacterial, fungal, or parasitic infections), cancers, or exposure to toxins. Thus, in one aspect, disclosed herein are methods of diagnosis or detection wherein the pathogenic infection is a viral infection selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A (including H1N1 or other Swine H1), Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

70. In one aspect, the disclosed methods of detecting or diagnosing a viral infection comprise contacting a tissue sample with an antibody against a viral antigen. Thus, also disclosed are methods wherein the antigen against which the detecting antibody is specific is a viral glycoprotein (GP), portal protein, tegument protein, capsid protein, DNA polymerase, RNA polymerase, reverse transcriptase, protease, integrase, DNA-binding protein, nucleoprotein (NP), nuclear matric protein, envelope protein (ENV), nuclear antigen, membrane protein, proteins encoded by viral early genes, group specific antigen (gag) protein, hemagglutinin (HA), neuraminidase (NA), or matrix protein. Specific examples of viral antigens include but are not limited to ENV, GP160 (HIV) GP120 (HIV), GP41 (HIV), EBNA-1, EBNA-2, EBNA-3, LMP-1, LMP-2, E1, E2, E3, E4, E5, E6, E7, NSP1, NSP2, NSP3, NSP4, NSP5, NSP10, NSP14, NSP15, NSP16, NSP29, G35P, G38P, G39P, zygocin protein, VP5 protein, 3AB protein, L4-22K protein, L4-100K protein, ORF 17 protein, S7 protein, S9 protein, S10 protein, HBXIP protein, UL3.5 protein, virus-infected-associated antigen protein, 3ABC protein, Cng protein, 2 BC protein, p58 protein, A40R protein, vpu protein, VPX protein, BPLF1 protein, NEF protein, SGTA protein, UL102 protein, p121 protein, VP35 protein, SPP1 Pac region protein, pX protein, N protein, agnoprotein, sigma NS protein, phage repressor proteins, U(S)3 protein kinase, ToxR protein, LexA protein, lambda CI repressor protein, Mu Ner protein, and Tat proteins.

71. Also disclosed are methods of detecting exposure to or diagnosis of a pathogenic infection wherein the antigen or pathogenic infection is a bacterial infection. For example, disclosed herein are methods wherein the bacterial infection is an infection with the bacteria selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In one aspect, the bacterial antigen against which the detecting antibody is specific can be a bacterial surface protein including but not limited to bacterial oligosaccharide, polysaccharide, or lipopolysaccharide; a protein associated with fimbrial structure and biogenesis, antimicrobial resistance, heavy metal transport, bacterial adhesion, extracytoplasmic substrate trafficking, or secreted hydrolases; exopolysaccharide; humic acid; N-acetylmuramic acid (NAM); N-acetylglucosamine (NAG); teichoic acids including ribitol teichoic acid and glycerol teichoic acid; O-antigen; Lipid A; pilin proteins; Porin; MA0829; or SbsB. In yet another aspect, the antigen can be a a component of a microbial biofilm, examples of which include but are not limited to exopolysaccharide, humic acid or other humic substances.

72. In another apect, disclosed herein are methods of diagnosing an infection or detecting antigenic exposure wherein the infection or antigen is a parasite. Examples of parasites that can be detected or diagnosed using the disclosed methods include parasites selected from the group consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica. Also disclosed are methods wherein the antigen is parasitophorous vacuole membrane-enclosed merozoite structures, galactose-inhibitable adherence protein, TSOL 16, MSP1, AMA1, Tryptophan rich antigens, MIC1, MAG1, or SAG1.

73. In another aspect, disclosed herein are methods of detecting exposure to a fungus or diagnosing a fungal infection wherein the fungi is selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata. It is understood and herein contemplated that fungal antigen against which the detecting antibody is specific can be Dse1, Int1, glucuronoxylomannan capsular polysaccharide, mannose polymers (mannan), galactomannan, Asp f 16 and Asp f 9, O-glycosylhydroases, β-endoglucanases, CRH-like proteins, Enolase, pyruvate decarboxylase, aldolase, pyruvate carboxylase, transketolase, phosphoglucomutase, HSP 30, 60, 80 and 90, AHP1, Elongation factor 1, Leishmanial elongation factor 4a, Phosphoglucomutase, Ribosomal L10 protein, PEP2, formate dehydrogenase, Histone H3, or Chitin.

74. Also disclosed herein are methods of detecting exposure to an antigen, wherein the antigen is derived from or is a toxin selected form the group consisting of Abrin, Conotoxins Diacetoxyscirpenol Bovine spongiform encephalopathy agent, Ricin, Saxitoxin, Tetrodotoxin, epsilon toxin, Botulinum neurotoxins, Shigatoxin, Staphylococcal enterotoxins, T-2 toxin, Diphtheria toxin, Tetanus toxoid, and pertussis toxin.

1. Immunoassays and Fluorochromes

75. Each of the detection and diagnostic methods described above utilize immunodetection through the use of label antibodies. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

76. In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

77. Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

78. As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

79. Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy₇; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP(Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP(S65T); GFP red shifted (rsGFP); GFP wild type’ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin 5; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof

80. A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

81. The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

82. Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

83. As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

84. Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-genrating molecule or moiety, allowing detection of the tertiary immune complexes thus formed. This system can provide for signal amplification.

85 Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

86. Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand Immunosensors allow the easy investigation of kinetic interactions and, with the advent of lower-cost specialized instruments, may in the future find wide application in immunoanalysis.

87. Immunohistochemistry and flow cytometry allow for the direct visualization of internal and external antigenic determinants through the binding of antibodies. It is contemplated herein that the disclosed antibodies can be used to detect the presence of an antigen intracellularly and thereby provide a diagnosis or indicate antigenic exposure. Additionally, the antibodies herein can be used in a research capacity to determine intracellular protein expression comprising administering to a cell a labeled IgG antibody to a protein, wherein the method does not comprise permeablizing the cell prior to administration of the antibody.

88. Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

89. One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

90. For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

91. Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

92. Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

93. Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

94. Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

95. Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

96. At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

97. Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

98. Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

99. Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Bioinvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

100. The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

101. Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

102. Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

103. An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

104. Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

105. Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

106. For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

107. As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

108. A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

E. Methods of Using the Compositions as Research Tools

109. The disclosed compositions can be used in a variety of ways as research tools. For example, the disclosed antibodies, being internalized through FcRn can be used to study the intracellular protein expression, such as, for example, IFN-γ or other cytokine expression in activated T cells. Thus, disclosed herein are methods of determining intracellular protein expression comprising administering to a cell a labeled IgG antibody to a protein, wherein the method does not comprise permeablizing the cell prior to administration of the antibody.

110. The disclosed antibodies and compositions can be used as discussed herein as either reagents in micro arrays or as reagents to probe or analyze existing microarrays. The disclosed compositions can be used in any known method for isolating or identifying single nucleotide polymorphisms. The antibodies and compositions can also be used in any known method of screening assays, related to chip/micro arrays. The antibodies and compositions can also be used in any known way of using the computer readable embodiments of the disclosed antibodies and compositions.

111. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

112. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### F. Examples

113. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### FcRn-Mediated IgG Neutralization of Influenza Virus

Transcytosis of the FcRn-IgG complex can be faithfully recapitulated in polarized Madin-Darby canine kidney (MDCK) cells stably transfected with rat FcRn and 132m (MDCK-FcRn). Additionally, MDCK is a classic model cell line for replicating influenza virus. Y8 mAb can only detect PR8 HA in conformational forms induced by an acidic pH. MDCK-FcRn was used as a cell-based model system. Y8 mAb or an irrelevant IgG was added to the basolateral chamber of MDCK-FcRn to initiate transcytosis. Subsequently, PR8 virus was added to the apical side to initiate infection. Viral yields were measured in the apical medium 24 h later by a 50% tissue culture infective dose (TCID₅₀) assay. The results showed that mAb Y8 reduced the yield of PR8 virus approximately 100-fold, but not in an MDCK-vector or IgG control monolayer (FIG. 1A). The extent of viral replication was further assessed by examining the expression of the influenza nucleoprotein (NP) gene. FcRn-mediated transcytosis of Y8 IgG, but not control IgG, significantly reduced the expression level of NP gene. These data further show that the intracellular inhibition of virus replication is dependent on the transepithelial flux of IgG.

Two additional experiments show that the intracellular neutralization of influenza virus by Y8 mAb in MDCK-FcRn cells was dependent on FcRn-mediated IgG transcytosis. First, MDCK cells expressing a chimeric FcRn and GFP are unable to transcytose IgG. Y8 mAb added to the basolateral chamber of MDCK-FcRn-GFP monolayers did not significantly reduce virus titers in comparison with those of control IgG-treated cells (FIG. 1B). In contrast, MDCK-FcRn cells produced significantly fewer viral progeny when incubated basolaterally with Y8 mAb.

Second, the virus titers in MDCK-FcRn/IgG cells that were pretreated with nocodazole reached comparable levels to those observed in untreated MDCK-vector cells. Thus, the intracellular neutralization of virus by Y8 mAb is dependent on FcRn-mediated IgG transport by polarized epithelial cells.

### 2. Example 2

### Colocalization of FcRn, Virus, and IgG in Endosomal Compartment

FcRn binding to IgG and Y8 mAb binding to PR8 virions both occur only at acidic pH. Acidic conditions also cause fusion between the endosomal membrane and the viral envelope. Y8 mAb transports into these endosomal compartments by FcRn, which interact with virus particles endocytosed from apical epithelial surfaces. To test this, confocal analysis of confluent MDCK-FcRn cells that were incubated with Y8 mAb and further infected with biotin-labeled PR8 virus was performed. The staining appeared in punctuate and vesicular patterns. Pair-wise colocalization of PR8 virus or FcRn with Y8 mAb showed significant colocalization in all cases. Furthermore, both Y8 mAb and PR8 virus colocalized with the early endosomal marker EEA1 in three-color confocal experiments. Most importantly, the colocalization results were confirmed by staining MDCK-FcRn cells that were inoculated basolaterally with Y8 mAb and apically infected with PR8. A Z-stack reconstructed view showed that the colocalization only occurred on the apical sides, demonstrating that Y8 IgG had been transported from the basolateral to apical domain.

### 3. Example 3

### Y8 mAb Neutralizes Viral Replication by Blocking Trafficking of Influenza vRNPs to the Nucleus

When influenza particles are endocytosed into endosomes, the acidic pH triggers fusion between the viral envelope and the endosomal membranes and release of yRNPs into the cytoplasm, with subsequent travel to the nucleus to initiate replication. The Y8 mAb acts by preventing viral envelope fusion with the endosomal membrane, preventing the trafficking of vRNPs to the nucleus. To test this, infected cells were stained with mAb anti-EEA1, an early endosome marker, or anti-NP protein to visualize vRNP trafficking to the nucleus. PR8 NP proteins were detected in the nucleus in control IgG-treated cells 1 h after infection, but not in the Y8 mAb-treated cells. Interestingly, the overall density of NP staining was significantly increased in control IgG-treated cells. NP was made rapidly in cells that were infected with this amount of virions, so the observed staining represents newly synthesized NP.

To further investigate the fate of virus particles, anti-lysosome-associated membrane glycoprotein-2 (LAMP-2), a lysosomal marker, and anti-NP mAbs were used to follow virion trafficking to nuclear or lysosomal sites. Transport of the virus particles to the lysosomes was negligible in control IgG-treated MDCK-FcRn cells during the incubation periods indicated. However, the colocalization of LAMP-2 and virus particles became more prominent at 45 min in Y8 mAb-treated cells, showing that this antibody promotes the trafficking of virus particles into the lysosomes. Pearson correlation coefficient analysis indicated a significant colocalization of viral NP protein with endosomal, lysosomal, and nucleus markers. Taken together, these data support that the Y8 mAb prevents influenza virus entry into the nucleus, by retaining the virus in endocytic compartments and by inhibiting the fusion of virus envelope and endosomal membranes, ultimately resulting in the delivery of these particles to lysosomes for degradation.

### 4. Example 4

### Prophylactic Efficacy of Y8 mAb Against PR8 Influenza Challenge in Vivo

Given FcRn expressed in the airway and mediated IgG transport across the airway mucosal barrier, it was of interest to know whether passive transfer of Y8 mAb confers protection from PR8 infection in mice. Groups of five WT and five FcRn-KO mice each received 100 μg of purified Y8 mAb via an intraperitoneal injection. Control groups of WT mice received isotype-matched IgG or sterile PBS solution. All mice were intranasally challenged 4 h later with a lethal dose (500 pfu) of PR8 virus. Because the serum half-life of IgG is greatly reduced in FcRn-KO mice, the FcRn KO mice were injected daily with 25-57.5 μg of Y8 mAb to compensate for IgG degradation. This supplementation strategy was first confirmed by injecting biotin-labeled IgG in a pilot experiment. In this way, the concentrations of Y8 mAb were expected to be similar between WT and FcRn-KO mice. Survival rates (FIG. 2A) and body weight losses (FIG. 2B) were then monitored. All WT animals that received Y8 mAb survived, whereas only 40% of the animals in the FcRn-KO group survived (P<0.05). The majority of animals that received irrelevant IgG or PBS solution died of infection within 6 d after challenge. Therefore, the administration of Y8 mAb in the WT mice was clearly associated with a survival benefit compared with control animals. Although the FcRn-KO mice receiving the Y8 mAb showed a trend toward increased survival, the increase was not significantly different from control animals. In addition, WT animals treated with Y8 mAb did not significantly lose body weight, whereas the mean weight loss in the control group was approximately 30% by the time the mice died or were euthanized (P<0.01). FcRn-KO mice that received Y8 mAb showed similar decreases in body weight as the control animals, with a 25% mean weight loss (FIG. 2B). In addition, all animals were assessed for viral load in the lungs at day 1 (FIG. 2C) or day 5 (FIG. 2D) after infection, after necropsy. The levels of virus in the lungs of WT mice, but not FcRn-KO mice treated with Y8 mAb, were 2.5 to 3 log10 TCID₅₀ lower than that in the control group (both P<0.01).

Pathological results were in accordance with the findings described earlier. No lesions were present in the lungs of mock-infected mice. In H36-4 mAb-treated animals, the loss of infectivity attributable to the combined inhibition of attachment and inhibition of fusion was sufficient to account for the extent of neutralization caused by relatively low concentrations of H36-4 mAb. WT animals that received Y8 mAb showed much less pulmonary pathology, such as edema or hemorrhagic appearance, or showed such lesions at a lower grade of severity, compared with control antibody- or PBS solution-treated animals: Examination of lungs in mice that received Y8 mAb on day 6 or 8 after infection revealed that mice did not develop apparent inflammatory changes although a slightly increased lymphocytic perivascular cuffing was observed. Examination of lungs in mice that received H36-4 mAb on day 6 or 8 after infection revealed a similar level of resolution. In contrast, FcRn KO mice that received Y8 developed peribronchiolar pneumonia that increased in severity, and a necrotizing bronchitis and bronchiolitis also appeared at this time point. Mice that received PBS solution and irrelevant IgG had continued peribronchiolar pneumonia and necrotizing bronchiolitis at day 6 after infection; the pneumonia was more widespread. The unprotected animals all died at day 6 to 7 after infection. Overall, these findings show that prophylactically administered Y8 mAb confers protection against lethal PR8 challenge, prevents mortality and viral replication, and reduces pulmonary pathology in an FcRn-dependent manner.

### 5. Example 5

### Discussion

IgG is the predominant Ig isotype present in the lungs. In the context of influenza virus, passive immunization by vertical acquisition or passive transfer demonstrates a clear role for virus-specific murine or humanized IgG antibodies in prophylaxis and therapy in both animal models as well as in infant humans. However, the precise cellular mechanisms by which these antibodies protect against viral infection and/or propagation remain elusive. Although the direct neutralization of viral particles is believed to be the primary function of antibodies in antiviral immunity, IgG is also efficient at fixing complement and binding to Fc receptors on cells. Indeed, “normeutralizing” antibody-dependent cellular cytotoxicity has been demonstrated in viral infection. However, these functions all require extracellular interactions, which do not occur between Y8 mAb and HA because this antibody is unable to bind HA at neutral pH. Herein, it is shown that anti-influenza IgG antibodies, traditionally considered to be normeutralizing IgG, are in fact capable of blocking viral infection in polarized epithelial cells via a mechanism that is dependent on FcRn-mediated transport of IgG.

To directly determine whether Y8 interferes within influenza infection, the intracellular neutralizing potential of Y8 mAb was evaluated by mimicking the mucosal epithelial barrier in vitro. Y8, but not control IgG, significantly reduced PR8 viral replication, showing that the blocking of viral replication by Y8 is dependent on transepithelial transcytosis of IgG by FcRn. Furthermore, Y8 colocalized with virions and FcRn inside endosomes, consistent with the intracellular colocalization of these proteins. Most importantly, Y8 mAb did not bind the PR8 virus at neutral pH, excluding the possibility that the Y8 neutralized virus in an extracellular environment. Therefore, the Y8 mAb interrupted viral replication during its encounter with viral particles in acidic endosomal compartments. The capacity of Y8 mAb to inhibit PR8 virus replication and reduce lung inflammation was further examined when Y8 mAb was administered to WT and FcRn-KO mice. Y8 mAb in WT mice provided strong protection from lethality, prevented weight loss, provided a significant reduction in pulmonary virus titers, and largely reduced virus-induced inflammation in the lungs. However, it should be noted that Y8 conferred some (albeit significantly less) protection from postinfection lethality and weight loss in FcRn-KO mice. This FcRn-independent effect is a result of fluid-phase uptake of the Y8 mAb into cells and endosomal entry in vivo. Overall, these results show a mechanism in which FcRn mediates the intracellular transport of anti-influenza IgG antibodies for endosomal neutralization sites in polarized epithelial cells.

An acidic pH aids FcRn binding to Y8 and for Y8 to interact with HA. Y8 mAb was shown to bind to internalized virus but not to virus absorbed to the cell surface. This finding is consistent with the fact that Y8 can bind to HA only following conformational changes caused by a low pH such as those that occur inside endosomes. Y8 is therefore sterically blocking an interaction between the endosomal membrane and a region of the influenza HA responsible for fusion. As such, FcRn organize IgG in the endosome in an orientation that facilitates the interaction with viral HA. Alternatively, FcRn simply increase the endosomal concentrations of IgG to levels that more effectively block viral fusion.

FcRn-mediated transport of IgG can be divided into several steps: IgG pinocytosis from the basolateral membrane to basolateral recycling endosomes, translocation from basolateral early endosomes to apical recycling endosomes (ARE), and finally, IgG recycling between the ARE and the apical plasma membrane. Therefore, transcytosis results in the accumulation of FcRn/Y8 mAb in the ARE. Intracellular neutralization results from the fusion of transcytotic vesicles containing Y8 mAb with vesicles containing endocytosed virions, showing that Y8 mAb blocks acid-induced fusion of the viral and endosome membranes required for vRNP entry into the cytoplasm and nucleus. Disclosed herein, inhibition of this fusion process was strongly shown by the fact that NP antigen from Y8 IgG-neutralized virus, unlike that of normeutralized virus, did not accumulate in the nucleus; instead, it was enriched in the lysosomes. Live cell imaging analyses of endothelial cells provides strong evidence that FcRn traffic its ligands to the lysosomes. FcyRI and FcyRIII engagement by IgG-bacteria immune complexes target intracellular bacteria to lysosomes in macrophages for degradation by a process strictly dependent on protein kinases involved in FcR intracellular signaling. Although these mechanisms are found in endothelial cells or macrophages, it is interesting to determine whether similar intracellular signaling and trafficking pathways operate in polarized epithelial cells to target antibody-coated viruses to lysosomes. Furthermore, although a highly pH-dependent mAb was used to demonstrate intracellular neutralization, a pH independent IgG that binds HA at a location that prevents a conformational change required for fusion functions in both extracellular neutralization and, upon encountering virus within endosomes, intracellular neutralization.

It is intriguing that Y8 mAb binds to the globular but not the fusion domain of the stalk region of influenza HA. Membrane fusion mediated by the influenza HA requires the concerted action of at least HA trimmers. By binding to low pH-induced monomeric HA molecules, Y8 mAb prevents a structural transition of HA required for fusion. Thus, Y8 mAb neutralizes intracellularly because it blocks fusion and egress from endosomes, resulting in the transport of virions to the lysosome for destruction. Other IgG antibodies with a broader spectrum of action or directed against the HA stalk regions containing the fusion domain work similarly, or even more effectively, by FcRn-dependent intracellular neutralization mechanisms at the mucosal surface. For example, antibodies can broadly recognize a highly conserved influenza virus epitope in the stalk regions of influenza HA; a vaccine based on the conserved HA stalk domain provided full protection against death and partial protection against disease following lethal viral challenge. Thus, heterotypic immunity involves several distinct immunological pathways, and the results herein illustrate that FcRn-mediated IgG transcytosis contributes to an intracellular neutralization mechanism.

The current paradigm for antibody-mediated mucosal immunity is that polymeric IgA receptor-mediated transcytosis of dimeric IgA releases secretory IgA into mucosal secretions by proteolytic cleavage. This transport process makes it possible for secretory IgA to block the binding of viruses to their entry receptors on the cell surface and to neutralize intracellular viruses. Disclosed herein, FcRn-mediated IgG transcytosis provides a mechanism for eliminating intracellular pathogens without destroying epithelial integrity (FIG. 3). This protective mechanism is determined by intracellular interactions between IgG and viral proteins enabled by the FcRn-mediated transport of IgG, by the specificity of the IgG for a particular viral component, and by the life cycle of the virus within mucosal epithelial cells. Similar intracellular neutralization mechanisms are applicable for HIV as well as bacteria. Recently, a cytosolic IgG receptor, tripartite motif-containing 21, bound and targeted incoming antibody-virus complexes to the proteasome via its E3 ubiquitin ligase activity. FcRn efficiently delivers IgG to intracellular vesicles. Thus provides an endosomal route for access to this cytosolic receptor.

### 6. Example 6

### Characterization of Influenza PR8-Specific Y8 mAb

Mouse IgG2a Y8 mAb binds to the globular domain of HA. Its cognate epitope is located at the interface of adjacent subunits. For this reason, Y8 mAb can bind influenza virus PR8 HA monomers but not native trimers. The Y8 mAb was further characterized in comparison with another HA-specific neutralizing mAb, H36-4, which can bind HA native trimers. Confluent MDCK cells were infected with influenza virus for 1.5 h at 4° C. to allow virus attachment to the cell surface and then at 37° C. for 30 min to permit viral endocytosis. The monolayers were stained with Y8 or H36-4 mAb with or without permeabilizing the cells. H36-4 mAb incubation resulted in a granular appearance of fluorescence staining; in contrast, antibody Y8 remained unreactive with virus particles adsorbed to the cell surface. H36-4, but not Y8 mAb, can react with surface virus. When the infected cells were warmed to 37° C. for 30 min before staining, both the H36-4 and Y8 mAbs reacted with the virus particles in permeabilized cells, as shown by the presence of discrete fluorescent spots in the cytoplasm, showing the Y8 mAb only recognizes intracellular HA. To further evaluate the difference of binding activity between the Y8 and H36-4 mAb, influenza virus PR8 was incubated with Y8 or H36-4 mAb in pH 5.0 or 7.4 buffer, readjusted to pH 7.4, and followed by HI assay. Treatment of purified influenza virus at pH 5.0 leads to irreversible conformational alterations in HA proteins. H36-4 mAb had potent HI activity at acidic and neutral pH; however, the Y8 mAb had HI activity only at an acidic pH. These results demonstrate that, unlike H36-4 mAb, Y8 mAb can only detect PR8 HA in conformational alterations induced by an acidic pH. Therefore, the pH sensitivity of Y8 mAb provides a unique tool to investigate the potential of FcRn-mediated IgG transport to block viral infection in epithelial cells.

### 7. Example 7

### Neutralization of Influenza PR8 Virus by Y8 mAb Is Dependent on IgG Transcytosis

Nocodazole, a reversible inhibitor of microtubule polymerization, has been shown to efficiently block IgG transcytosis. Preincubation of MDCK-FcRn monolayers with 33 μm nocodazole abolished the apically directed transport of Y8 IgG in a transcytosis assay. Likewise, the virus titers in MDCK-FcRn/IgG cells that were pretreated with nocodazole reached comparable levels as those observed in untreated MDCK-vector cells. Although it was not tested directly, MDCK cells return to a normal cell state following nocodazole removal, thus, the 2-h nocodazole pretreatment does not significantly impact normal viral replication during the subsequent 24-h incubation.

### 8. Example 8

### In Vivo Transcytosis of Y8 mAb

By Western blot analysis, FcRn is highly expressed in respiratory epithelial cells. It was subsequently tested whether murine FcRn can mediate IgG transport across the airway mucosal barrier in WT vs. FcRn-KO mice. Biotin-IgG was administered into WT (100 mg) or FcRn-KO (200 μg) mice i.p. The rationale for injecting twofold more IgG into FcRn-KO mice is both endogenous and injected IgG exhibit fast clearance in these mice; thus, more IgG is required in FcRn-KO mice to obtain comparable exposure levels. As a specificity control, 200 mg of chicken IgY-biotin was also injected into the WT mice. Lung bronchoalveolar lavage (BAL) samples were taken 24 h after each injection and subjected to avidin blot analysis. IgG was detected in the BAL of WT mice. The failure to detect IgG in FcRn KO mice and chicken IgY in WT mice is consistent with specific transport of IgG by across alveolar tissue by FcRn in vivo.

### 9. Example 9

### Expressions of FcRn and pIgR in Mouse Airway Tissues

Although both IgA and IgG are transcytosed by Fc receptors, IgG is the major Ig isotype detected in human bronchoalveolar fluid. The differential expression levels of FcRn and pIgR, which transcytoses IgA through the polarized epithelial cells, explains this discrepancy in the BAL. The levels of mouse FcRn and pIgR expression in the lung and trachea of adult mice was examined by immunofluorescence staining and Western blot analysis. The liver and intestine were used as controls. FcRn was detected in the epithelial cells of both trachea and lung. However, the pIgR was barely detectable in the lung alveolar epithelial cells, although it was detected in the bronchial and tracheal epithelial cells. The pIgR was abundant in the epithelial cells of the liver and small intestine. However, mouse FcRn was detected in only the liver. These results explain why large amounts of IgG, but not IgA, appear in the BAL. This observation has biological significance for antibody-mediated immunity against respiratory infections in lung tissues.

### 10. Methods

a) Cells, Antibodies, and Mice.

Madin-Darby canine kidney (MDCK) type II cell line was a gift from Keith Mostov. Cells were grown in DMEM complete medium supplemented with 10 mM Hepes, 10% FCS, 1% L-glutamine, nonessential amino acids (Invitrogen), and 1% penicillin/streptomycin (Invitrogen Life Technologies). When necessary, media were also complemented with 400 μg/mL of G418 (Invitrogen). Cells were grown in 5% CO₂ at 37° C. mAb anti-EEA1 was obtained from BD Biosciences. Goat anti-mouse polymeric IgA receptor (pIgR) was from R&D Systems. Mouse anti-canine LAMP-2 was from AbD Serotec, and anti-3-tubulin antibody was obtained from the Developmental Studies Hybridoma Bank, developed under the auspices of the National Institute of Child Health and Human Development and maintained by the University of Iowa. ZO-1-specific antibody was obtained from Invitrogen. Alexa Fluor-conjugated 488, 555, and Alexa Fluor 633 goat anti-mouse or rabbit antibodies were purchased from Molecular Probes. Affinity-purified rabbit IgG against mouse FcRn was used. HRP-conjugated donkey anti-rabbit or rabbit anti-mouse antibody was purchased from Pierce Biotechnology Affinity-purified mouse IgG and chicken IgY were obtained from Rockland Immunochemicals. Sulfo-NHS-LC-biotin was from Pierce.

b) Virus and mAb.

Influenza A virus (strain A/Puerto Rico/8/1934 H1N1) was a gift from Peter Palese (Mount Sinai School of Medicine, New York, N.Y.). Influenza PR8 virus was grown in 10- to 11-d-old embryonated chicken eggs. Purification of the virus was performed by differential centrifugation and sedimentation through a sucrose gradient. PR8HA-specific hybridoma Y8(IgG2a) and H36-4 (IgG2a) were from Coriell Institute for Medical Research, and the NP-specific hybridoma HB-65(IgG2a) was from the American Type Culture Collection. Purification of mAb from cell culture medium was done by affinity chromatography using Protein A (Pierce), dialyzed against PBS solution and stored in PBS solution at −80° C. Influenza virus (2 mg/mL) was biotinylated by using 150 μM sulfo-NHS-SS-biotin (Pierce) according to manufacturer's instructions, and free biotin was completely removed by desalting column (Pierce).

c) SDS/PAGE, Western Blot, and Avidin Blot.

Protein concentrations were determined by Bradford assay (Bio-Rad Laboratories). Then, proteins or biotin-labeled proteins were resolved on a 12% SDS/PAGE gel under reducing conditions. Proteins were transferred onto a nitrocellulose membrane (Schleicher and Schuell). The membranes were blocked with 5% nonfat powered milk, probed separately with anti-FcRn, anti-pIgR, or 3-tublin Ab for 1 h, and followed by incubating with HRP-conjugated rabbit anti-mouse IgG, HRP-conjugated donkey anti-rabbit IgG or HRP-avidin, respectively. All blocking, incubating, and washing were performed in PBS solution with 0.05% Tween 20. Proteins were visualized by the ECL method (Pierce).

d) TCID₅₀ Assay and Hemagglutination Inhibition Assay.

TCID₅₀ was determined in MDCK cells. Samples were serially diluted 10-fold in Opti-MEM I (Invitrogen). MDCK cells were plated 1 d before PR8 infection in 96-well plates.

MDCK confluent monolayers were then infected with the diluted virus for 1 h at 37° C. Infected cells were subsequently washed and incubated with fresh medium supplemented with 1 mg/mL TPCK-trypsin (Sigma) for 72 h. Supernatant were collected and the endpoint viral titer was determined by a hemagglutination assay.

The antiviral activity of PR8 HA specific mAb was measured by standard hemagglutination inhibition (HI) assay with minor modifications. Approximately four hemagglutination units of PR8 viruses were incubated at pH 5.0 or pH 7.4 Opti-MEM for 2 h at 4° C. Spin desalting columns were used to exchange the buffer to neutral pH (7.4). Y8 or H36 mAbs were serially diluted 10-fold in V-bottom 96-well plates and incubated for 1 h at room temperature with viruses treated at different pH values. Subsequently, 1% chicken red blood cells were added and incubated for 30 min at room temperature. The highest serum dilution that inhibited hemagglutination was considered the HI titer of the mAb.

e) In Vitro and in Vivo Transcytosis.

IgG transcytosis in MDCK monolayers was measured. MDCK cells expressing rat FcRn or FcRn-GFP, and MDCK-vector control, were grown onto 0.4-um pore size trans-well filter inserts (Corning Costar) to form a monolayer exhibiting transepithelial electrical resistances (300 Ω/cm²). Transepithelial electrical resistance was measured by using a volt-ohm meter equipped with planar electrodes (World Precision Instruments). Monolayers were equilibrated in Hanks balanced salt solution. IgG, IgG-biotin, or IgY-biotin (400 mg/mL) were applied to the basolateral compartment in pH 7.4 serum-free DMEM (Invitrogen) supplemented with 10 mM Hepes, 10 mM sodium pyruvate, 1% L-glutamine, 1% nonessential amino acids, and 1% penicillin/streptomycin, and incubated for 2 h at 37° C. Transported proteins were sampled from the apical chamber and analyzed by SDS/PAGE under reducing conditions. Proteins were visualized by Western blot-ECL or avidin blot-ECL analysis. For in vivo IgG or IgY transport, 200 mg of biotinylated mouse IgG or chicken IgY in 100 μL of PBS solution were injected i.p. into mice. Lung lavages were collected 24 h later. The transported IgG-biotin or IgY-biotin antibody was analyzed by SDS/PAGE and Western blot-ECL or avidin blot-ECL analysis (Pierce).

f) Intracellular Neutralization of PR8 Virus by Y8 mAb.

Y8 IgG (400 mg/mL) or an irrelevant murine IgG2a antibody was applied to the lower compartment when MDCK-FcRn or MDCK-vector cell monolayers become polarized on insert filters. Cells were incubated with IgG antibody for 2 h at 37° C. Subsequently, PR8 virus (100 pfu/cell) was inoculated into the apical chamber for 1.5 h at 4° C.; then, cells were warmed to 37° C. for an additional 45 min to allow infection. Inserts were completely washed to remove the residual antibody or virus. Cells were incubating for additional 24 h at 37° C., at which time the apical supernatants were removed. The apical supernatants were tested for virus titers by TCID₅₀.

g) Nocodazole Treatment.

MDCK transfectants (1×10⁵) were seeded onto the transwell to allow polarization. Cells were preincubated with or without nocodazole (33 μm) for 2 h; nocodazole was then removed from the chambers. Y8 mAb (400 mg/mL) was subsequently added into the basolateral chamber to allow transport for 2 h. PR8 virus was added to the apical chamber for 45 min to allow infection. Cells were completely washed to remove the IgG or virus and incubated for an additional 24 h at 37° C. The amount of PR8 virus in the apical medium was analyzed by a TCID₅₀ assay.

h) RT-PCR Analysis.

For total RNA extraction, cells were pelleted and resuspended in TRIzol reagent (Invitrogen Life Technologies). The influenza PR8 NP gene was amplified by primers (5′-AT-CATGGCGTCTCAAGGCAC-3′(SEQ ID NO: 1), 5′-TCCTGTATATAGGTC-CTC-3′ (SEQ ID NO: 2)) with an One-Step RT-PCR kit (Qiagen). The RNA was also amplified by using GAPDH-specific primers (5′-GGAG-AAAGCTGCCAAATATG-3′ (SEQ ID NO: 3), 5′-TACCAGGAAATGAGCT-TGAC-3′ (SEQ ID NO: 4)) as an internal control to monitor the quality of the RNA purification and cDNA synthesis. The PCR products were analyzed by 1.5% agarose gel electrophoresis and stained with ethidium bromide.

i) Immunofluorescence and Confocal Microscopy.

Immunofluorescence staining of cells or frozen tissue sections was performed. Briefly, cells were cultivated on coverslips for 24 h and subsequently incubated with Y8 for 2 h at 37° C. Next, antibody-treated cells were incubated with biotin-labeled virus for 30 min. The cells were rinsed in PBS solution, fixed in 3.7% paraformaldehyde (Sigma) in PBS solution for 30 min at 4° C., and quenched with 10% glycine for 10 min. After two washings with PBS solution, the coverslips were permeabilized in PBS solution containing 0.2% Triton X-100 for 20 min. The frozen tissues were embedded in optimal cutting temperature media, serially sectioned, fixed in acetone for 5 min at −20° C., and air-dried for 30 min. Both cells and tissue sections were blocked with 10% normal goat serum for 30 min and stained with affinity-purified primary antibodies in PBS solution with 0.05% Tween 20 with 3% BSA for 1 h, followed by Alexa Fluor 555- or Alexa Fluor 488-conjugated anti-IgG antibodies of the corresponding species in blocking buffer. Biotinylated virus was detected by using streptavidin conjugates labeled with Alexa Fluor 488 (Molecular Probes). After each step, cells were washed at least three times with PBS solution containing 0.05% Tween-20. Coverslips were mounted on slides with ProLong antifade reagent (Molecular Probes) and examined by using a Zeiss LSM 510 confocal fluorescence microscope. The images were processed by using LSM Image Examiner software (Zeiss). Quantitative colocalization measurement was performed by using Zeiss LSM 510 Examiner Software. Pearson correlation coefficient was calculated for describing the colocalization correlation of the intensity distributions between two channels.

j) Analysis of Intracellular Distribution of Nucleoprotein Protein After PR8 Infection in Presence or Absence of Y8 mAb.

MDCK-FcRn cells were cultivated on coverslips for 24 h. Cells were treated with 400 μg/mL Y8 mAb or isotype-matched IgG for 1 h. Cells were infected with PR8 virus at a multiplicity of infection of 100 pfu/cell at 4° C. for 1.5 h. After they were washed with cold PBS solution three times, cells were shifted to 37° C. in culture medium and collected at 10, 30, 45, 60, 120, or 240 min. Cells were stained with primary anti-EEA1, LMAP-2, and anti-NP mAb. Other staining procedures are the same as the described for immunofluorescence and confocal microscopy. Quantitative co-localization measurements were performed by using Zeiss LSM 510 Examiner Software. Pearson correlation coefficients were calculated for describing the colocalization correlation of intensity distributions between the two channels. In the quantitative experiment with MDCK-FcRn cells, 10 cells per view field were analyzed. P<0.05 was considered as significant.

k) Passive Protection of WT and FcRn KO Mice Against PR8 Virus by mAb.

Groups of five mice were injected i.p. with 100 μL of PBS solution with 100 μg Y8 or mouse IgG 4 h before challenge to allow distribution and equilibration of antibody to all tissues before virus inoculation. One group of five mice was mock-immunized with PBS solution following the same schedule. Mice were inoculated with 500 pfu PR8 viruses intranasally under an anesthesia induced with 100 μL of 40 mg/mL tribromoethanol (Avertin; Sigma). Mice were kept on their backs under the influence of anesthesia for 45 min to allow infection. Mice were monitored for 10 d for illness and death. Body weight changes were recorded on a daily basis. For virus titration in the lung, viruses were inoculated into MDCK cells and cultured for 3 d, and TCID₅₀ values were measured.

l) Pathology.

To assess pulmonary inflammation after PR8 virus infection, lungs were taken from experimental mice to examine the gross pathologic changes after biopsy. Lungs were also immediately placed in 10% neutral buffered formalin and sent to American HistoLabs, where they were embedded in paraffin and stained with H&E to visualize cellular inflammation. Slides were coded and read “blind.”

### G. References

Bächi T, Gerhard W, Yewdell J W (1985) Monoclonal antibodies detect different forms of influenza virus hemagglutinin during viral penetration and biosynthesis. J Virol 55:307-313.
Baker K, et al. (2009) Immune and non-immune functions of the (not so) neonatal Fc receptor, FcRn. Semin Immunopathol 31:223-236.
Barbey-Martin C, et al. (2002) An antibody that prevents the hemagglutinin low pH fusogenic transition. Virology 294:70-74.
Bomsel M, et al. (1998) Intracellular neutralization of HIV transcytosis across tight epithelial barriers by anti-HIV envelope protein dlgA or IgM. Immunity 9:277-287.
Burns J W, Siadat-Pajouh M, Krishnaney A A, Greenberg H B (1996) Protective effect of rotavirus VP6-specific IgA monoclonal antibodies that lack neutralizing activity. Science 272:104-107.
Casadevall A (1998) Antibody-mediated protection against intracellular pathogens. Trends Microbiol 6:102-107.
Corthesy B, Kraehenbuhl J-P (1999) Antibody-mediated protection of mucosal surfaces. Curr Top Microbiol Immunol 236:93-111.
Danieli T, Pelletier S L, Henis Y I, White J M (1996) Membrane fusion mediated by the influenza virus hemagglutinin requires the concerted action of at least three hemagglutinin trimers. J Cell Biol 133:559-569.
Ekiert D C, et al. (2009) Antibody recognition of a highly conserved influenza virus epitope. Science 324:246-251.
Florese R H, et al. (2006) Evaluation of passively transferred, normeutralizing antibody-dependent cellular cytotoxicity-mediating IgG in protection of neonatal rhesus macaques against oral SIVmac251 challenge. J Immunol 177:4028-4036.
Forthal D N, Moog C (2009) Fc receptor-mediated antiviral antibodies. Curr Opin HIV AIDS 4:388-393.
Gan Z, Ram S. Vaccaro C, Ober R J, Ward E S (2009) Analyses of the recycling receptor, FcRn, in live cells reveal novel pathways for lysosomal delivery. Traffic 10:600-614.
Israel E J, Wilsker D F, Hayes K C, Schoenfeld D, Simister N E (1996) Increased clearance of IgG in mice that lack beta 2-microglobulin: Possible protective role of FcRn. Immunology 89:573-578.
Jerdeva G V, et al. (2010) Comparison of FcRn- and pIgR-mediated transport in MDCK cells by fluorescence confocal microscopy. Traffic 11:1205-1220.
Joller N, et. al. (2010) Antibodies protect against intracellular bacteria by Fc receptor-mediated lysosomal targeting. Proc Natl Acad Sci USA 107:20441-20446.
Kaetzel C S, Robinson J K, Chintalacharuvu K R, Vaerman J P, Lamm M E (1991) The polymeric immunoglobulin receptor (secretory component) mediates transport of immune complexes across epithelial cells: A local defense function for IgA. Proc Natl Acad Sci USA 88:8796-8800.
Li Z, et al. (2011) Transfer of IgG in the female genital tract by MHC class 1-related neonatal Fc receptor (FcRn) confers protective immunity to vaginal infection. Proc Natl Acad Sci USA 108:4388-4393.
Mallery D L, et al. (2010) Antibodies mediate intracellular immunity through tripartite motif-containing 21 (TRIM21). Proc Natl Acad Sci USA 107:19985-19990.
Mazanec M B, Kaetzel C S, Lamm M E, Fletcher D, Nedrud J G (1992) Intracellular neutralization of virus by immunoglobulin A antibodies. Proc Natl Acad Sci USA 89:6901-6905.
Momose F, Kikuchi Y, Komase K, Morikawa Y (2007) Visualization of microtubule-mediated transport of influenza viral progeny ribonucleoprotein. Microbes Infect 9:1422-1433.
Nicholls J M, Chan R W, Russell R J, Air G M, Peiris J S (2008) Evolving complexities of influenza virus and its receptors. Trends Microbiol 16:149-157.
Ober R J, Martinez C, Vaccaro C, Zhou J, Ward E S (2004) Visualizing the site and dynamics of IgG salvage by the MHC class 1-related receptor, FcRn. J Immunol 172:2021-2029.
Raghavan M, Bonagura V R, Morrison S L, Bjorkman P J (1995) Analysis of the pH dependence of the neonatal Fc receptor/immunoglobulin G interaction using antibody and receptor variants. Biochemistry 34:14649-14657.
Renegar K B, Small P A, Jr., Boykins L G, Wright P F (2004) Role of IgA versus IgG in the control of influenza viral infection in the murine respiratory tract. J Immunol 173:1978-1986.
Reuman P D, Paganini C M, Ayoub E M, Small P A, Jr. (1983) Maternal-infant transfer of influenza-specific immunity in the mouse. J Immunol 130:932-936.
Reynolds H Y (1987) Identification and role of immunoglobulins in respiratory secretions. Eur J Respir Dis Suppl 153:103-116.
Rojas R, Apodaca G (2002) Immunoglobulin transport across polarized epithelial cells. Nat Rev Mol Cell Biol 3:944-955.
Roopenian D C, Akilesh S (2007) FcRn: The neonatal Fc receptor comes of age. Nat Rev Immunol 7:715-725.
Roopenian D C, et al. (2003) The MHC class I-like IgG receptor controls perinatal IgG transport, IgG homeostasis, and fate of IgG-Fc-coupled drugs. J Immunol 170:3528-3533.
Rust M J, Lakadamyali M, Zhang F, Zhuang X (2004) Assembly of endocytic machinery around individual influenza viruses during viral entry. Nat Struct Mol Biol: 11:5670573.
Simmons C P, et al. (2007) Prophylactic and therapeutic efficacy of human monoclonal antibodies against H5N1 influenza. PLoS Med 4:e178.
Spiekermann G M, et al. (2002) Receptor-mediated immunoglobulin G transport across mucosal barriers in adult life: Functional expression of FcRn in the mammalian lung. J Exp Med 196:303-310.
Steel J, et al. (2010) Influenza virus vaccine based on the conserved hemagglutinin stalk domain. mBio 1:pii:e00018-10.
Sui J, et al. (2009) Structural and functional bases for broad-spectrum neutralization of avian and human influenza A viruses. Nat Struct Mol Biol 16:265-273.
Tesar D B, Tiangco N E, Bjorkman PJ (2006) Ligand valency affects transcytosis, recycling and intracellular trafficking mediated by the neonatal Fc receptor. Traffic 7:1127-1142.
Tucker S P, Compans R W (1993) Virus infection of polarized epithelial cells. Adv Virus Res 42:187-247.
Ward E S, Ober R J (2009) Chapter 4: Multitasking by exploitation of intracellular transport functions the many faces of FcRn. Adv Immunol 103:77-115.
White J M, Wilson I A (1987) Anti-peptide antibodies detect steps in a protein conformational change: Low-pH activation of the influenza virus hemagglutinin. J Cell Biol 105:2887-2896.
Ye L, et al. (2008) The MHC class II-associated invariant chain interacts with the neonatal Fc gamma receptor and modulates its trafficking to endosomal/lysosomal compartments. J Immunol 181:2572-2585.
Yewdell J W, Gerhard W, Bachi T (1983) Monoclonal anti-hemagglutinin antibodies detect irreversible antigenic alterations that coincide with the acid activation of influenza virus A/PR/834-mediated hemolysis. J Virol 48:239-248.
Yoshida M, et al. (2006) Neonatal Fc receptor for IgG regulates mucosal immune responses to luminal bacteria. J Clin Invest 116:2142-2151.

What is claimed is:

1. An antibody specific for a non-surface expressed antigen or an antigenic determinant that is only accessible to an antibody through a conformational change of the antigen.

2. The antibody of claim 1, wherein the antibody is a neutralizing antibody.

3. The antibody of claim 1, wherein the antibody is specific for an antigen present on mucosal surfaces of a subject or on a pathogen that has infected mucosal surfaces of a subject.

4. The antibody of claim 1, wherein the antibody has an IgG isotype.

5. The antibody of claim 1, wherein the antigen is present in or on the surface of a pathogen or encoded by a pathogen.

6. The antibody of claim 1, wherein the antigen is a viral antigen from a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A (including H1N1 or other Swine H1), Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

7. The antibody of claim 6, wherein the antigen is a viral glycoprotein (GP), portal protein, tegument protein, capsid protein, DNA polymerase, RNA polymerase, reverse transcriptase, protease, integrase, DNA-binding protein, nucleoprotein (NP), nuclear matric protein, envelope protein (ENV), nuclear antigen, membrane protein, proteins encoded by viral early genes, group specific antigen (gag) protein, hemagglutinin (HA), neuraminidase (NA), or matrix protein.

8. The antibody of claim 1, wherein the antigen is a bacterial antigen from a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

9. The antibody of claim 8, wherein the antigen is bacterial oligosaccharide, polysaccharide, or lipopolysaccharide; a protein associated with fimbrial structure and biogenesis, antimicrobial resistance, heavy metal transport, bacterial adhesion, extracytoplasmic substrate trafficking, or secreted hydrolases; exopolysaccharide; humic acid; N-acetylmuramic acid (NAM); N-acetylglucosamine (NAG); teichoic acids including ribitol teichoic acid and glycerol teichoic acid; O-antigen; Lipid A; pilin proteins; Porin; MA0829; or SbsB.

10. The antibody of claim 1, wherein the antigen is a parasitic antigen from a parasite selected from the group consisting of Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

11. The antibody of claim 10, wherein the antigen is parasitophorous vacuole membrane-enclosed merozoite structures, galactose-inhibitable adherence protein, TSOL 16, MSP1, AMA1, Tryptophan rich antigens, MIC1, MAG1, or SAG1.

12. The antibody of claim 1, wherein the antigen is a fungal antigen from a fungus selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

13. The antibody of claim 12, wherein the antigen is Dse1, Int1, glucuronoxylomannan capsular polysaccharide, mannose polymers (mannan), galactomannan, Asp f 16 and Asp f 9, O-glycosylhydroases, β-endoglucanases, CRH-like proteins, Enolase, pyruvate decarboxylase, aldolase, pyruvate carboxylase, transketolase, phosphoglucomutase, HSP 30, 60, 80 and 90, AHP1, Elongation factor 1, Leishmanial elongation factor 4a, Phosphoglucomutase, Ribosomal L10 protein, PEP2, formate dehydrogenase, Histone H3, or Chitin.

14. The antibody of claim 1, wherein the antigen is encoded by a cancer.

15. The antibody of claim 14 wherein the cancer is selected from the group of cancers consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumors, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

16. The antibody of claim 15, wherein the antigen is c-S is, PDGF, CSF-1, EGF, PMA, IGF-1, IGF-2, IL-1, IL-2, IL-6, IL-8, estrogens, androgens, VEGF, FGF, Src-family proteins, Syk-ZAP-70, BTK, pp 125, E6 and E7 from Human papillomavirus, JAK family proteins, Raf, cyclin-dependent kinases, protein kinase A (PKA), protein kinase B (AKT), protein kinase C (PKC), phosphatidylinositol 3-kinase (PI3K), mTOR, mitogen-activated protein kinases (MAPKs), ERK1, ERK2, ERK3, ERK4, ERK5, ERK6, ERK7, JNKs, p38, MKK1, MKK2, RSK kinase, ASK1, TAK1, MLK3, TAOK1, Ca2+/calmodulin-dependent protein kinases (CaM Kinase), ribosomal S6 kinase, IRAK1, Ras, Rho, Rab, Arf, Ran, Ral, Rac, myc or c-Myc, a STAT family protein, a HOX family protein, NF-κB, AP-1, SP1, NF-1, Oct-1, ATF/CREB, C/EBP, Elk-1, c-Jun, c-Fos or steroid recpetors.

17. The antibody of claim 1, wherein the antigen is an allergen selected from the allergens from group consisting of house Mites Mite, House Dust Dermatophagoides farinae Mite, House Dust Dermatophagoides pteronyssinus Mite, Acarus siro Food/Storage Mite, House Dust Blomia tropicalis Mite, Storage Chortoglyphus arcuates Mite, House Dust Euroglyphus maynei Mite, Lepidoglyphus Food/Storage destructor Mite, Tyrophagus Food/Storage putrescentiae Mite, House Dust Glycyphagus domesticus Venoms Bumble Bee Bombus spp. Venom European Hornet Vespa crabro Venom Honey Bee Apis mellifera. Venom Mixed Hornet Dolichovespula Venom spp Mixed Paper Polistes spp. Wasp Venom Mixed Yellow Vespula spp. Jacket Venom White (bald)-Dolichovespula faced Hornet maculate Venom Yellow Hornet Dolichovespula Venom arenaria Insects Ant, Carpenter Camponotus pennsylvanicus Ant, Fire Solenopsis invicta Ant, Fire Solenopsis richteri Cockroach, Periplaneta American Americana Cockroach, Blattella German germanica Cockroach, Blatta orientalis Oriental Horse Fly Tabanus spp. House Fly Musca domestica Mayfly Ephemeroptera spp. Mosquito Culicidae sp. Moth Heterocera spp. Epithelia, Dander, Hair & Feathers Canary Feathers Serinus canaria Cat Epithelia Felis catus (domesticus) Cattle Epithelia Bos Taurus Chicken Feathers Gallus gallus (domesticus) Dog Epithella, Canis familiaris Mixed Breeds Duck Feathers Anas platyrhynchos Gerbil Epithelia Meriones unguiculatus Goat Epithelia Capra hircus Goose Feathers Anser domesticus Guinea Pig Cavia porcellus Epithelia (cobaya) Hamster Epithelia Mesocricetus auratus Hog Epithelia Sus scrofa Horse Epithelia Equus caballus Mouse Epithelia Mus musculus Parakeet Feathers Psittacidae spp. Pigeon Feathers Columba fasciata Rabbit Epithelia Oryctolagus cuniculus Rat Spithelia Rettus norvegicus Wool, Sheep Ovis aries Dander Cat Felis catus dander/Antigen (domesticus) Dog Dander, Canis familiaris Mixed-Breed Poodle Dander Canis familiaris Fungi Acremonium Cephalosporium strictum acremonium Alternaria Alternaria alternate tenuis Aspergillus Aspergillus amstelodami glaucus Aspergillus flavus Aspergillus furmigatus Aspergillus nidulans Aspergillus niger Aspergillus terreus Aspergillus versicolor Aureobasidium Pullularia pullulans pullulans Bipolaris Drechslera sorokiniana sorokiniana, Helminthosporium sativum Botrytis cinerea Candida albicans Chaetomium globosum Cladosporium herbarum Cladosporium Hormodendrum sphaerospermum hordei Drechslere Curvularia spicifera spicifera Epicoccum Epicoccum nigrum purpurascens Epidermophyton floccosum Fusarium moniliforme Fusarium solani Geotrichum Oospora lactis candidum Gliocladium Gliocladium viride deliquescens Helminthosporium Spondylocladium solani atrovirens Microsporum Microsporum canis lanosum Mucor Mucor mucedo circinelloides f. circinelloides Mucor Mucor circinelloides f. racemosus lusitanicus Mucor plumbeus Mycogone perniciosa Neurospora Neurospora intermedia sitophila, Monilia sitophila Nigrospora oryzae Paecilomyces variotii Penicillium brevi-compactum Penicillium camembertii Penicillium chrysogenum Penicillium digitatum Penicillium expensum Penicillium notatum Penicillium roquefortii Phoma betae Phomma Phoma herbarum pigmentivora Rhigopus oryzae Rhizopus arrhizus Rhizopus Rhizopus stolonifer nigricans Rhodotorula Rhodotorula mucilaginosa rubra var. mucilaginosa Saccharomyces cerevisiae Scopulariopsis brevicaulis Serpula lacrymans Merulius lacrymans Setosphaeria Exserohilum rostrata rostratum, Helminthosporium halodes Stemphylium botryosum Stemphylium solani Trichoderma Trichoderma harzianum viride Trichophyton Trichophyton mentagrophytes interdigitale Trichophyton rubrum Trichothecium Cephalothecium roseum roseum Smuts Barley Smut Ustilago nuda Bermuda Grass ustilago Smut cynodontis Corn Smut Ustilago maydis Johnson Grass Sporisorium Smut cruentum Oat Smut Ustilago avenae Wheat Smut Ustilago tritici Grass Pollens Bahia Paspalum notatum Bermuda Cynodon dactylon Blue, Canada Poa compressa Brome, Smooth Bromus inermis Canary Phalaris arundinacea Corn Zea mays Couch/Quack Elytrigia repens (Agropyron repens) Johnson Sorghum, halepense Kentucky Blue Poa pratensis Meadow Fescue Festuca pratensis (elatior) Oat, Cultivated Avena sativa Orchard Dactylis glomerata Red Top Agrostis gigantean (alba) Rye, Cultivated Secale cereale Rye, Giant Wild Leymus (Elymus) condensatus Rye, Italian Lolium perenne ssp. multiflorum Rye, Perennial Lolium perenne Sweet Vernal Anthoxanehum odoratum Timothy Phleum pratense Velvet Holcus lanatus Wheat, Cultivated Triticum aestivum Wheatgrass, Elymus Western (Agropyron) smithii Weed Pollens Allscale Atriplex polycarpa Baccharis Baccharis halimifolia Baccharis Baccharis sarothroides Burrobrush Hymenoclea salsola Careless Weed Amaranthus hybridus Cocklebur Xanthium strumarium (commune) Dock, Yellow Rumex crispus Dog Fennel Eupatorium capillifolium Goldenrod Solidago spp. Hemp, Western Amaranthus Water tuberculatus (Acnida tamariscina) Iodine Bush Allenrolfea occidentalis Jerusalem Oak Chenopodium botrys Kochia/Firebush Kochia scoparia Lambs Quarter Chenopodium album Marsh Elder, Iva xanthifolia Burweed Marsh Elder, Iva angustifolia Narrowleaf Marsh Elder, Iva annua Rough (ciliata) Mexican Tea Chenopodium ambrosioides Mugwort, Artemisia Common vulgaris Mugwort, Artemisia Darkleaved ludoviciana Nettle Urtica dioica Palmer's Amaranthus Amaranth palmeri Pigweed, Amaranthus Redroot/Rough retroflexus Pigweed, Spiny Amaranthus spinosus Plantain, English Plantago lanceolata Poverty Weed Iva axillaris Quailbrush Atriplex lentiformis Rabbit Bush Ambrosia deltoidea Ragweed, Desert Ambrosia dumosa Ragweed, False Ambrosia acanthicarpa Ragweed, Giant Ambrosia trifida Ragweed, Short Ambrosia artemisiifolia Ragweed, Slender Ambrosia confertiflora Ragweed, Ambrosia Southern bidentata Ragweed, Ambrosia Western psilostachya Russian Thistle Salsola kali (pestifer) Sage, Coastal Artemisia californica Sage, Pasture Artemisia frigida Sagebrush, Artemisia Common tridentate Saltbush, Annual Atriplex wrightii Shadscale Atriplex confertifolia Sorrel, Red/Sheep Rumex acetosella Wingscale Atriplex canescens Wormwood, Artemisia annua Annual Tree Pollens Acacia Acacia spp. Alder, European Alnus glutinosa Alder, Red Alnus rubra Alder, Tag Alnus incana ssp. rugosa Alder, White Alnus rhombifolia Ash, Arizona Fraxinus velutina Ash, Green/Red Fraxinus pennsylvanica Ash, Oregon Fraxinus latifolia Ash, White Fraxinus americana Aspen Populus tremuloides Bayberry Myrica cerifera Beech, American Fagus grandifolia (americana) Beefwood/Austral Casuarina ian Pine equisetifolia Birch, Betula lenta Black/Sweet Birch, European Betula pendula White Birch, Red/River Betula nigra Birch, Spring Betula occidentalis (fontinalis) Birch, White Betula populifolia Box Elder Acer negundo Cedar, Japanese Cryptomeria japonica Cedar, Mountain Juniperus ashei (sabinoides) Cedar, Red Juniperus virginiana Cedar, Salt Tamarix gallica Cottonwood, Populus Black balsamifera ssp. trichocarpa Cottonwood, Populus Eastern deltoides Cottonwood, Populus Fremont fremontii Cottonwood, Rio Populus Grande wislizeni Cottonwood, Populus Western monilifera (sargentii) Cypress, Arizona Cupressus arizonica Cypress, Bald Taxodium distichum Cypress, Italian Cupressus sempervirens Elm, American Ulmus americana Elm, Cedar Ulmus crassifolia Elm, Siberian Ulmus pumila Eucalyptus Eucalyptus globulus Hackberry Celtis occidentalis Hazelnut Corylus americana Hazelnut, Corylus European avellana Hickory, Pignut Carya glabra Hickory, Carya ovata Shagbark Hickory, Carya laciniosa Shellbark Hickory, White Carya alba Juniper, Oneseed Juniperus monosperma Juniper, Pinchot Juniperus pinchotii Juniper, Rocky Juniperus Mountain scopulorum Juniper, Utah Juniperus osteosperma Juniper, Western Juniperus occidentalis Locust Blossom, Robinia Black pseudoacacia Mango Blossom Mangifera indica Maple, Coast Acer macrophyllum Maple, Red Acer rubrum Maple, Silver Acer saccharinum Maple, Sugar Acer saccharum Melaleuca Melaleuca quinquenervia (leucadendron) Mesquite Prosopis glandulosa (julifiora) Mulberry, Paper Broussonetia papyrifera Mulberry, Red Morus rubra Mulberry, White Morums alba Oak, Quercus Arizona/Gambel gambeiji Oak, Black Quercus velutina, Oak, Bur Quercus macrocarpa Oak, California Quercus Black kelloggii Oak, California Quercus Live agrifolia Oak, California Quercus lobata White/Valley Oak, English Quercus robur Oak, Holly Quercus ilex Oak, Post Quercus stellata Oak, Red Quercus rubra Oak, Scrub Quercus dumosa Oak, Virginia Quercus Live virginiana Oak, Water Quercus nigra Oak, Western Quercus White/Gany garryana Oak, White Quercus alba Olive Olea europaea Olive, Russian Elaeagnus angustifolia Orange Pollen Citrus sinensis Palm, Queen Arecastrum romanzoffianum (Cocos plumosa) Pecan Carya illinoensis Pepper Tree Schinus molle Pepper Schinus Tree/Florida terebinthifolius Holly Pine, Loblolly Pinus taeda Pine, Eastern Pinus strobus White Pine, Longleaf Pinus palustris Pine, Ponderosa Pinus ponderosa Pine, Slash Pinus elliottii Pine, Virginia Pinus virginiana Pine, Western Pinus monticola White Pine, Yellow Pinus echinata Poplar, Lombardy Populus nigra Poplar, White Populus alba Privet Ligustrum vulgare Sweet Gum Liquidambar styraciflua Sycamore, Platanus Eastern occidentalis Sycamore, Platanus Oriental orientalis Sycamore, Platanus Western racemosa Sycamore/London Platanus Plane acerifolia Walnut, Black Juglans nigra Walnut, Juglans California Black californica Walnut, English Juglans regia Willow, Arroyo Salix lasiolepis Willow, Black Salix nigra Willow, Pussy Salix discolor Flowers: Wild & Cultivated Daisy, Ox-Eye Chrysanthemum leucanthemum Dandelion Taraxacum officinale Sunflower Helianthus annuus Cultivated Farm Plant Pollens Alfalfa Medicago sativa Castor Bean Ricinus communis Clover, Red Trifolium pratense Mustard Brassica spp. Sugar Beet Beta vulgaris Plant Food Almond Prunus dulcis Apple Malus pumila Apricot Prunus armeniaca Banana Musa paradisiaca (sapientum) Barley Hordeum vulgare Bean, Lima Phaseolus lunatus Bean, Navy Phaseolus vulgaris Bean, Pinto Phaseolus sp. Bean, Red Kidney Phaseolus sp. Bean, Phaseolus String/Green vulgaris Blackberry Rubus allegheniensis Blueberry Vaccinium sp. Broccoli Brassica oleracea var. botrytis Buckwheat Fagopyrum esculentum Cabbage Brassica oleracea var. capitata Cacao Bean Theobroma cacao Cantaloupe Cucumis melo Carrot Daucus carota Cauliflower Brassica oleracea var. botrytis Celery Apium graveolens var. dulce Chemy Prunus sp. Cinnamon Cinnamomum verum Coffee Coffee arabica Corn Zea mays Cranberry Vaccinium macrocarpon Cucumber Cucumis sativus Garlic Allium sativum Ginger Zingiber officinale Grape Vitis sp. Grapefruit Citrus paradisi Hops Humulus lupulus Lemon Citrus limon Lettuce Lactuca sativa Malt Mushroom Agaricus campestris Mustard Brassica sp. Nutmeg Myristica fragrans Oat Avena sativa Olive, Green Olea europaea Onion Allium cepa var. cepa Orange Citrus sinensis Pea, Blackeye Vigna unguiculata Pea, Green Pisum sativum (English) Peach Prunus persica Pear Pyrus communis Pepper, Black Piper nigrum Pepper, Green Capsicum annuum var. annuum Pineapple Ananas comosus Potato, Sweet Ipomoea batatas Potato, White Solanum tuberosum Raspberry Rubus idaeus var. idaeus Rice Oryza sativa Rye Secale cereale Sesame Seed Sesamum orientale (indicum) Soybean Glycine max Spinach Spinacia oleracea Squash, Yellow Cucurbita pepo var. melopepo Strawberry Fragaria chiloensis Tomato Lycopersicon esculentum (lycopersicum) Turnip Brassica rapa var. rapa Vanilla Bean Vanilla planifolia Watermelon Citrullus lanatus var. lanatus Wheat, Whole Triticum aestivum Fish & Shellfish Bass, Black Micropterus sp. Catfish Ictalurus punctatus Clam Mercenaria mercenaria Codfish Gadus morhua Crab Callinectes sapidus Flounder Platichthys sp. Halibut Hippoglossus sp. Lobster Homarus americanus Mackerel Scomber scombrus Oyster Crassostrea virginica Perch Sebastes marinus Salmon Salmo salar Sardine Clupeiformes Scallop Pectan magellanicus Shrimp Penaeus sp. Trout, Lake Salvelinus sp. Tuna Fish Thunnus sp Animal Foods Beef Bos taurus Lamb Ovis aries Pork Sus scrofa Poultry Products Chicken Gallus gallus Egg, Chicken, Gallus gallus. White Egg (Gallus gallus), Yolk (Meleagris gallopavo), Casein, Brazil Nut Bertholletia excels, Cashew Nut Anacardium occidentale, Coconut Cocos nucifera, Filbert/Hazelnut Corylus Americana, Peanut Arachis hypogaea, Pecan Carya illinoensis, Walnut, Black Juglans nigra Walnut, English Juglans regia, and latex.

18. A composition comprising an antibody of claim 1.

19. A method of treating or inhibiting a disease or condition comprising administering to a subject one or more antibodies of claim 1.

20. A method of diagnosing a disease or condition or detecting exposure to an antigen in a subject comprising obtaining a tissue sample from the subject and contacting the tissue with one or more antibodies of claim 1, wherein the one or more antibodies comprise a detectable label, wherein detection of the one or more antibodies indicates the subject has the disease or condition or has been exposed to the pathogen.
